(19) United States
(12) Patent Application Publication
(10) Pub. No.: US 2017/0273301 A1
(43) Pub. Date: Sep. 28, 2017

(54) Hyperprotonation Compositions And Methods Of Use For Cleaning, Disinfection, And Sterilization

(71) Applicant: Keith Benson, Reston, VA (US)

(72) Inventor: Keith Benson, Reston, VA (US)

(21) Appl. No.: 15/469,077

(22) Filed: Mar. 24, 2017

Related U.S. Application Data
(60) Provisional application No. 62/312,524, filed on Mar. 24, 2016.

(51) Int. Cl. A01N 37/02; A01N 25/30

(52) U.S. Cl. CPC A01N 37/02; A01N 25/30

(57) ABSTRACT

Compositions and methods for cleaning, disinfection, sterilization, and decontamination of surfaces and objects are provided. In particular, a hyperprotonation composition is described that comprises a surfactant, one or more emulsifiers, a biocide, and a weak acid and is effective to disrupt both the microbial biofilm defenses as well as the microbes within. Methods of applying the hyperprotonation compositions to contaminated surfaces, equipment, fabrics, food, and human or animal tissue to disrupt the microbial biofilms and eradicate the microbes within are also disclosed.

This application claims the benefit of U.S. Provisional Application No. 62/312,524, filed Mar. 24, 2016, the entire content of which is incorporated by reference herein.

### FIELD

The field of the invention relates generally to compositions and methods for cleaning, disinfecting, and sterilizing surfaces, equipment, living tissue, and other media. In particular, the invention provides hyperprotonation compositions for the disruption of microbial biofilms to allow and enhance access of antimicrobial agents to the microbes contained therein.

### BACKGROUND

It is generally understood that cleaning and disinfecting compositions for surfaces, equipment, and human skin and tissues do not achieve complete eradication of microbe colonies. Common cleaning and disinfecting compositions based on active ingredients such as potassium hydroxide (e.g., LYSOL) and sodium hypochlorite (e.g., CLOROX) are publicly marketed as “killing 99.9% of viruses and bacteria” when applied. However, those claims are based on results of laboratory planktonic testing procedures in which the composition is applied directly to microorganisms in suspension.

Extensive research has shown that the planktonic testing environment used for assessing the efficacy of common cleaners and disinfectants does not accurately represent results in the actual environments in which microorganisms thrive. Indeed, microorganisms such as Pseudomonas aeruginosa, Bacillus anthracis, Escherichia coli, Staphylococcus aureus, Proteus vulgaris, and Listeria monocytogenes typically colonize within physical matrices known as biofilms. Biofilms are matrix-enclosed accumulations of microorganisms such as bacteria (with their associated bacteriophages), fungi, protozoa, and viruses. While biofilms are rarely composed of a single cell type, there are common circumstances where a particular cellular type predominates. The non-cellular components are diverse and may include carbohydrates; both simple and complex; proteins, including polypeptides; lipids; and lipid complexes of sugars and proteins (lipopolysaccharides and lipoproteins).

Bacterial biofilms are comprised of an extracellular matrix that is produced by bacteria once they attach to a surface, which helps to protect the microbes from immune cells and antimicrobial agents. Since efficacy of antimicrobial agents (e.g., antibiotics, antiseptics, disinfectants, and antiviral compounds) is compromised by the extracellular biofilm matrix, strategies to disrupt the biofilm and expose the microorganisms within can be helpful in increasing the activity level of antimicrobial agents and thus reducing the concentration of such agents needed to make an effective composition.

The architecture of biofilms is not simply an aggregation. Rather, biofilms are distinct communities that acquire new features and functions beyond those of their individual members. Because of the properties provided by microorganisms in a biofilm, microbes in biofilms are typically less susceptible to antibiotics, antimicrobials, biocides, and antiviral agents. In some cases, bacteria in a biofilm can be up to 4,000 times more resistant (i.e., less susceptible) than the same organism in a planktonic state.

The role of biofilms is discussed in U.S. Patent Pub. No. 2014/0275267, which notes that:
bacterial organisms which actively populate these common surfaces may form organized communities called biofilms. Bacterial cells forming these biofilm communities assume a biological phenotype that is markedly different than their corresponding planktonic (non-surface attached, or free-swimming) bacterial analogs . . . . Biofilms are a special form of contamination that have been shown to require as much 1000 times the dose of routine biocides in order to eradicate the microorganism contained within, as compared to planktonic forms.

The presence of extracellular polymeric substances (EPS) on the outer surface of biofilms is known to reduce the efficacy of cleaning, disinfecting, and sterilizing compositions. As noted above, EPS creates physical and chemical defenses that protect the microorganisms within the matrix, resulting in substantial survival rates and regrowth. When commonly used cleaning and disinfecting compositions are applied, portions of microbial colonies that are protected by the EPS then reproduce rapidly after application. Thus, it is typical with respect to a disinfectant advertised as “killing 99.9% of viruses and bacteria” (based on applications in solution using planktonic testing), that in the real world applications where EPS is prevalent, they will kill much lower percentages, and colonies will regrow rapidly. Laboratory tests have shown that some products claimed to have a 99.9% kill rate actually kill less than 30% of the microorganisms in biofilms.

Moreover, real world contamination often includes combinations of different types of microorganisms within biofilm-protected colonies (poly-microbial contamination). Cleaners and disinfectants currently in general use may be effective only against certain microorganisms, and not others. The commonly used tests assess effectiveness against mono-microbial test parameters, not typical poly-microbial contamination scenarios.

Cleaners and disinfectants currently on the market are significantly ineffective in the presence of biofilms. One aspect of the problem is that biofilms have a wide range of pH. It had previously been viewed that pH was homogenous across microorganism environments at around pH 5 to 7. Recent studies, however, have shown that the pH range of biofilms is broader, ranging from about 3 to 8. In addition, biofilm pH is both variable and dynamic. In reacting to contact with certain treatment compositions, the pH of biofilm may change. The prior art has generally considered the problem of biofilms as a steady-state issue, assuming no variation, and not testing for such variation. Thus, the industry has been focused on applying compositions without addressing the true nature of the problem. This problem creates particular challenges with respect to compositions including weak acids, which ultimately rely on the process of protonation. Dynamic pH changes in biofilm can result in equilibrium in pH at the contact layer with weak acid solutions resulting in pH below the titration point.

Another aspect of the problem is that biofilms provide physical and chemical defenses for the microorganisms that must be breached in order to disrupt the living organism within. These defenses can include both the EPS layer of the biofilm and an inner layer of lipopolysaccharides (LPS). For example, studies have been cited suggesting that the intact LPS layer of enterobacteriaceae protected those organisms from anti-bacterial compositions.

Thus, microorganisms in biofilm colonies can be considered to have at least two distinct defense mechanisms: (1) the mechanism whereby the pH of the biofilm results in a change in pH at the composition contact layer that may be within the titration or inactivation point of the active ingredient, or to equilibrium; and (2) physical protections afforded by the EPS and LPS layers.

Current cleaners and disinfectants are not generally suited for addressing a broad spectrum range of various types of microorganisms. One problem is that there is such a variation of chemical composition and physical nature of microbes, that in order to have a broad-spectrum attack, it is necessary to identify and address the lowest common denominator or common defenses. Variations include physical and chemical composition of EPS/LPS, particularly in gram-negative bacteria, which can operate to make the penetration of biocides to be ineffective. A composition seeking to be effective on a broad spectrum basis must adequately address these variations.

Examples of microorganisms that are not effectively eradicated with current cleaners and disinfectants include the following:
Staphylococcus aureus is a gram-positive bacterium that is a common cause of infections. The organism is ubiquitous, with estimates of 30-40% of humans being colonized on mucosal surfaces. Illnesses caused by the organism range from benign infections, such as furuncles, to life-threatening illnesses, such as toxic shock syndrome (TSS)
Bacillus anthracis is a gram-positive rod that, through production of a cell surface capsule and other molecules and exotoxins, can cause serious illnesses. Such illnesses include skin, gastrointestinal, and pulmonary anthrax. This organism is characterized as a “category A select agent.”
Methicillin-resistant Staphylococcus aureus (MRSA) is a bacterium responsible for several difficult-to-treat infections in humans. It is also called oxacillin-resistant Staphylococcus aureus (ORSA). MRSA is any strain of S. aureus that has developed, through the process of natural selection, resistance to beta-lactam antibiotics, which include the penicillins (e.g., methicillin, dicloxacillin, nafcillin, oxacillin, etc.) and the cephalosporins.

A primary chemical interaction which can result in the breakdown of biofilms, LPS, and microorganisms, is protonation. Protonation is a fundamental chemical reaction and is a step in many stoichiometric and catalytic processes. Protonation and deprotonation occur in most acid-base reactions and are the core of most acid-base reaction theories.

For a given compound, protonation occurs at the point when the active molecule will donate the relevant proton, which is called the titration point. For example, the necessity of achieving the requisite composition pH and amine oxide protonation is discussed in U.S. Pat. No. 6,255,270, which discloses liquid cleaning compositions that include an amine oxide detergent, a quaternary disinfectant (quat), an acidifying agent, an effective amount of an electrolytic disinfecting booster, and an aqueous carrier.

The failure of certain cleaners and disinfectants to break down EPS and LPS defenses and eradicate microorganisms can result from insufficient or ineffective protonation. One problem is that protonation may require maintaining a sufficient difference in pH between the composition donating the protons and that of the surfactant layer in proximity to the microorganisms. In the event that the pH of the solution and the contact biomass is below the titration point for the active ingredient, protonation will reduce or cease and no longer effectively break down EPS and LPS defenses or disrupt the microorganisms therein.

Even where EPS and LPS defenses can be breached, it also is important to apply effective antimicrobial and biocidal substances to the microbes within. For example, as explained in U.S. patent Pub. No. 2013/0281532:
[m]ost bacterial pathogens initiate human illnesses from intact or damaged mucosal or skin surfaces. Many of these pathogens are acquired from other persons or animals, from endogenous sources, or from a myriad of environmental sources. Once in humans, pathogens colonize surfaces primarily as biofilms of organisms, defined as thin-films of organisms attached to host tissues, medical devices, and other bacteria through complex networks of polysaccharides, proteins, and nucleic acids. These bacteria may also exist as planktonic (broth) cultures in some host tissue environments, such as the bloodstream and mucosal secretions. Similarly, these potential pathogens may exist as either biofilms or planktonic cultures in a myriad of non-living environments.

US Pub. No. 2013/0281532 discusses compositions of glycerol monolaurate (GML), a naturally occurring glycerol-based compound that has previously been shown to have antimicrobial, anti-viral, and anti-inflammatory properties, to be applied as a topical composition in treating microbial infections and illnesses. GML is one chemical within the broader family of glycerol monoesters (GMEs). The class of GME compositions, including GML, have in certain circumstances been demonstrated to have potent antibacterial activity against gram-positive microorganisms and Bacillus anthracis. U.S. Pub. No. 2013/0281532 discloses that:
unlike most antibiotics which have single bacterial targets for antibacterial activities, GML appears to target many bacterial surface signal transduction systems nonspecifically through interaction with plasma membranes. GML also inhibits exotoxin production by gram-positive bacteria at GML concentrations that do not inhibit bacterial growth. These properties are shared with the antibiotic clindamycin, a protein synthesis inhibitor. GML is also virucidal for enveloped viruses, apparently through its ability to interfere with virus fusion with mammalian cells, and through GML's ability to prevent mucosal inflammation required for some viruses to penetrate mucosal surfaces. Studies demonstrate that GML is bactericidal for aerobic and anaerobic gram-positive bacteria in broth and biofilm cultures, GML exhibits greater bactericidal activity than lauric acid, and all forms of GML exhibit antibacterial activity. Additionally, GML is bactericidal for gram-negative bacteria with LOS instead of LPS, but GML becomes bactericidal for naturally GML-resistant Enterobacteriaceae by addition of agents that disrupt the LPS layer. Gram-negative anaerobes are susceptible to GML. Pseudomonas aeruginosa appear to be the most resistant bacteria tested, but these organisms are killed by GML at pH 5.0-6.0.

U.S. Pub. No. 2013/0281532 describes other studies demonstrating that GML and other compounds within the family of GME have potent bactericidal activity against many microorganisms causing human illnesses, including gram-positive bacteria (notably, gram-positive cocci); anaerobes; pathogenic clostridia; Candida; Gardnerella vaginalis; Staphylococcus aureus; and Streptococcus agalactiae. This includes both aerobes and anaerobes, and gram-positive, gram-negative, and non-gram-staining bacteria.

US patent application no. 0281532 concluded that:
it is thought that GML inhibits microbial infection through one or more of several mechanisms that include, but are not limited to, direct microbial toxicity; inhibiting entry of the infectious microorganism into the vertebrate cell; inhibiting growth of the microorganism; inhibiting production or activity of virulence factors such as toxins; stabilizing the vertebrate cells; or inhibiting induction of inflammatory or immunostimulatory mediators that otherwise enhance the infectious process.

The class of GME compositions, including GML, have been demonstrated to have potent antibacterial activity, as explained in recent NIH research reports, but subject to important perceived limitations. Schlievert, et al. Glycerol Monolaurate Antibacterial Activity in Broth and Biofilm Cultures, 10.1371/journal.pone.0040 350 (2012). GML's biocidal effect is substantially increased in low pH. However, NIH's recent research believed that “it is unlikely that GML will be used as an antibacterial agent as suspended in aqueous solutions do to its solubility limit of 100 μg/ml in aqueous solutions at 37° C.”

Thus there remains a need in the art for effective compositions for reducing or disrupting a microbial biofilm's EPS and LPS defenses in order to effectively deliver biocidal agents to the microbial biomass for the cleaning, disinfection, and/or sterilization of surfaces, equipment, human skin, and other media which are contaminated with microorganisms, such as bacteria, viruses, yeasts, and molds.

### SUMMARY

Aspects of the present invention feature compositions that enhance the disruption of microbial biofilms and increase delivery of antimicrobial agents to the microbes within the microbial biofilms. In addition, provided herein are methods of applying the compositions for cleaning, disinfecting, or sterilizing a surface or object on which is disposed a microbial biofilm.

One aspect of the invention features a composition for cleaning, disinfecting, or sterilizing a surface or object on which is disposed a microbial biofilm, where the composition includes: (a) a cationic surfactant in an amount from about 1% w/v to about 5% w/v; (b) one or more emulsifying agents in an amount from about 0.5% w/v to about 5% w/v; (c) a biocide in an amount of at least about 0.1% w/v, provided that the biocide is a glycol monoester of the formula: R₁OCH₂(OR₂)CH₂OR₃ where R₁, R₂ and R₃ are individually H or a C6 to C22 acyl group; and (d) at least one weak acid in an amount from about 0.5% w/v to about 15% w/v, provided that the at least one weak acid has a pH is less than about 3.5 and the cationic surfactant has a pH of at least about 2 units greater than the first titration point pH of the at least one weak acid. Furthermore, when the composition is applied to the surface or object, a wetting layer is formed that increases protonation of water to produce hydronium and increases delivery of the hydronium and the biocide to the microbial biofilm thereby disrupting the microbial biofilm.

In one embodiment, the cationic surfactant is a fatty acid salt or a saponified organic acid, and the at least one weak acid is selected from the group consisting of ascorbic acid, salicylic acid, citric acid, lactic acid, malic acid, tartaric acid, and any combination thereof. In another embodiment, the cationic surfactant is potassium cocoate. In other embodiments, the one or more emulsifying agents are selected from the group consisting of sorbitan monolaurate, sodium stearoyl lactylate, polyoxyethylene (20) sorbitan monooleate, and any combination thereof. In a particular embodiment, the glycol monoester is selected from the group consisting of glycerol monocaprylate, glycerol monocaprate, glycerol monolaurate, glycerol monomyristate, and any combination thereof. In some embodiments, the composition is added to a cleaning formulation selected from the group consisting of toilet bowl cleaner, metal cleaner, metal brightener, rust stain remover, denture cleanser, metal descaler, general hard surface cleaner, and disinfectant.

Another aspect of the invention features a method for cleaning, disinfecting or sterilizing a surface or object on which is disposed a microbial biofilm. The method includes applying a hyperprotonation composition to the surface or object comprising the microbial biofilm. In this method, the hyperprotonation composition includes: (a) a cationic surfactant in an amount from about 1% w/v to about 5% w/v; (b) one or more emulsifying agents in an amount from about 0.5% w/v to about 5% w/v; (c) a biocide in an amount of at least about 0.1% w/v, provided that the biocide is a glycol monoester of the formula: R₁OCH₂(OR₂)CH₂OR₃ where R₁, R₂ and R₃ are individually H or a C6 to C22 acyl group; and (d) at least one weak acid in an amount from about 0.5% w/v to about 15% w/v, provided that the at least one weak acid has a pH less than about 3.5 and the cationic surfactant has a pH of at least about 2 units greater than the first titration point pH of the at least one weak acid. Furthermore, upon application of the composition on the surface or object comprising the microbial biofilm, a wetting layer is formed that increases protonation of water to produce hydronium and increases delivery of the hydronium and the biocide to the microbial biofilm thereby disrupting the microbial biofilm and cleaning, disinfecting, or sterilizing the surface or object.

In some embodiments of the method, (i) the cationic surfactant is a fatty acid salt or a saponified organic acid having a pH greater than about 8; (ii) the at least one weak acid is selected from the group consisting of ascorbic acid, salicylic acid, citric acid, lactic acid, malic acid, tartaric acid, and any combination thereof; (iii) the one or more emulsifying agents are selected from the group consisting of sorbitan monolaurate, sodium stearoyl lactylate, polyoxyethylene (20) sorbitan monooleate, and any combination thereof; and (iv) the glycol monoester is selected from the group consisting of glycerol monocaprylate, glycerol monocaprate, glycerol monolaurate, glycerol monomyristate, and any combination thereof.

In one embodiment, the surface or object is in a sports facility, fitness facility, stadium locker room, gymnasium, country club, restaurant, hospital, hotel, or university. In another embodiment, the surface or object is a fruit or vegetable. In yet another embodiment, the surface or object is sprayed with the hyperprotonation composition or immersed in the hyperprotonation composition. In some embodiments, the microbial biofilm comprises one or more microorganisms selected from the group consisting of gram positive bacterium, gram negative bacterium, virus, yeast, mold, and any combination thereof.

In an embodiment, the applying may include flood application, spray application, high pressure application, foam application, or clean-in-place application. In another embodiment, the applying is part of a sterilization sequence for medical devices. In yet other embodiments, the hyperprotonation composition is contacted with the surface or object for a period of time of about 30 seconds to about 5 minutes, and wherein the method further comprises rinsing the hyperprotonation composition off of the surface or object after the period of time.

In some embodiments, the surface or object is selected from the group consisting of a piece of equipment, fabric, countertop, wall, door, toilet, shower stall, bathtub, sink, and chair food, locker, locker room, gymnasium floor, and living tissue. In other embodiments, the surface or object is a living tissue, and the hyperprotonation composition further comprises a pharmacologically acceptable carrier. In still other embodiments, the applying of the hyperprotonation composition to the surface or object produces a stable emulsified mixture in accordance with the hydrophilic-lipophilic balance system.

Another aspect of the invention features a composition for producing a hydronium engine on a microbial biofilm. The composition includes a cationic surfactant, one or more emulsifying agents, a biocide of the formula R₁OCH₂(OR₂)CH₂OR₃ where R₁, R₂ and R₃ are individually H or a C6 to C22 acyl group, and at least one weak acid with a pH less than or equal to 3.5 and a first titration point that is at least about 2 units less than the pH of the cationic surfactant. Furthermore, upon application of the composition on the microbial biofilm, it produces an emulsion layer and a wetting layer. The wetting layer increases protonation of water from the weak acid in the emulsion layer to produce hydronium and increases delivery of the hydronium and the biocide to the microbial biofilm thereby disrupting the microbial biofilm.

In one embodiment of the composition: (a) the cationic surfactant comprises potassium cocoate in an amount from about 1% w/v to about 5% w/v; (b) the one or more emulsifying agents are selected from the group consisting of glycerol monocaprylate, glycerol monocaprate, glycerol monolaurate, glycerol monomyristate, and any combination thereof, and in an amount from about 0.5% w/v to about 5% w/v; (c) the biocide is selected from the group consisting of glycerol monocaprylate, glycerol monocaprate, glycerol monolaurate, glycerol monomyristate, and any combination thereof, and in an amount of at least about 0.1% w/v; and (d) the at least one weak acid is selected from the group consisting of ascorbic acid, salicylic acid, citric acid, lactic acid, malic acid, tartaric acid, and any combination thereof, and in an amount from about 0.5% w/v to about 15% w/v.

Other features and advantages of the invention will be understood by the detailed description, drawings and examples that follow.

### BRIEF DESCRIPTION OF THE DRAWINGS

The accompanying figures, which are incorporated in and constitute a part of this specification, illustrate several aspects and, together with the description, serve to explain the principles of the invention.

FIG. 1 is an illustration depicting the hyperprotonation layer at a microbial biofilm created by application of the compositions and systems of the invention. Three layers are depicted (from top to bottom of the illustration): (1) the emulsion, (2) the surfactant wetting layer, and (3) the microbial biomass. Lines between the three layers indicate (from top to bottom): the boundary layer created between the emulsion and the wetting layer, and the microbial biofilm. In the embodiment shown, the wetting layer is greater than pH 4.11, therefore above the lowest titration point of the citric acid disposed in the emulsion, causing titration and hyperprotonation through the wetting layer. Further, the titration event in the wetting layer does not consume the surfactant and therefore does not reach equilibrium, as would occur if there was direct contact with the biomass.

FIG. 2 is a graph depicting the hyperprotonation—pH balance and kill zone of an exemplary hyperprotonation composition. The y-axis indicates the weight percentage of citric acid, and the x-axis indicates the pH of the solution. In preferred embodiments, (1) the biocide (GME) concentration is greater than 500 micrograms per ml, (2) the surfactant concentration is greater than 0.5% w/v, (3) the steady state pH of the solution is not greater than the titration point of the acid, and (4) the pH of the surfactant mix (with emulsifier and GME) is at least 2 pH units higher than the lowest titration point of the acid.

FIG. 3 is a table depicting the effect of citric acid concentration on the change in pH of the surfactant and emulsifier composition for an embodiment of the invention. The composition of the exemplary hyperprotonation composition for the range of component values is balanced by distilled water (% w/v). The composition of GML in 0.50% emulsifiers is 750 μg/ml. The composition of GML in 0.75% emulsifiers is 1,125 μg/ml. The composition of GML in 1.00% emulsifiers is 1,500 μg/ml.

FIG. 4 is graph showing the log reduction of E. coli over time after contacting with an embodiment of the invention. The y-axis indicates the log reduction of E. coli, and the x-axis indicates the amount of time elapsed in minutes.

FIG. 5 is graph showing the log reduction of Salmonella spp. over time after contacting with an embodiment of the invention. The y-axis indicates the log reduction of Salmonella spp., and the x-axis indicates the amount of time elapsed in minutes.

FIG. 6 is graph showing the log reduction of S. aureus over time after contacting with an embodiment of the invention. The y-axis indicates the log reduction of S. aureus, and the x-axis indicates the amount of time elapsed in minutes.

FIG. 7 is graph comparing the log reduction of Salmonella spp. over time after contacting with an embodiment of a hyperprotonation composition (circle) as compared to benzalkonium chloride (triangle), bleach (diamond), and lye (square). The y-axis indicates the log reduction of Salmonella spp., and the x-axis indicates the amount of time elapsed in minutes.

### DETAILED DESCRIPTION

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Composition, formulation, and/or reaction components may have several known functions, but may be selected and identified for a particular function (e.g., a buffer). However, as one skilled in the art may appreciate, the component may be performing multiple functions within the composition, formulation, and or reaction (e.g., a surfactant may function as a wetting agent and as an emulsifier).

All percentages expressed herein are by weight of the total volume of the composition or mixture unless expressed otherwise. All ratios expressed herein are on a weight per volume (% w/v) or weight per total weight (% wt or wt %) basis as indicated.

Ranges may be used herein in shorthand, to avoid having to list and describe each value within the range. Any appropriate value within the range can be selected, where appropriate, as the upper value, lower value, or the terminus of the range.

As used herein, the singular form of a word includes the plural, and vice versa, unless the context clearly dictates otherwise. Thus, the references “a”, “an”, and “the” are generally inclusive of the plurals of the respective terms. For example, reference to “a method” or “a microbe” includes a plurality of such “methods”, or “microbes.” Likewise the terms “include”, “including”, and “or” should all be construed to be inclusive, unless such a construction is clearly prohibited from the context. Similarly, the term “examples,” particularly when followed by a listing of terms, is merely exemplary and illustrative and should not be deemed exclusive or comprehensive.

The term “comprising” is intended to include embodiments encompassed by the terms “consisting essentially of” and “consisting of”. Similarly, the term “consisting essentially of” is intended to include embodiments encompassed by the term “consisting of.”

The methods and compositions and other advances disclosed herein are not limited to particular equipment or processes described herein because such equipment or processes may vary. Further, the terminology used herein is for describing particular embodiments only and is not intended to limit the scope of that which is disclosed or claimed.

Unless defined otherwise, all technical and scientific terms, terms of art, and acronyms used herein have the meanings commonly understood by one of ordinary skill in the art in the field(s) of the invention, or in the field(s) where the term is used. Although any compositions, methods, articles of manufacture, or other means or materials similar or equivalent to those described herein can be used in the practice of the present invention, the preferred compositions, methods, articles of manufacture, or other means or materials are described herein.

The term “about” refers to the variation in the numerical value of a measurement, e.g., temperature, parts per million (ppm), pH, concentration, volume, etc., due to typical error rates of the device used to obtain that measure. In one embodiment, the term “about” means within 5% of the reported numerical value.

The term “antimicrobial” refers effectiveness in preventing, inhibiting, or arresting the growth or pathogenic effects of a microorganism.

The term “biocide” refers to a chemical substance or microorganism which can deter, render harmless, or exert a controlling effect on an organism by chemical or biological means. “Biocides” are commonly used in medicine, agriculture, forestry, and industry. Biocidal substances and products are also employed as anti-fouling agents or disinfectants under other circumstances: chlorine, for example, is used as a short-life biocide in industrial water treatment but as a disinfectant in swimming pools. Many biocides are synthetic, but a class of natural biocides are derived from, e.g., bacteria and plants. As used herein, “biocide” can refer to a pesticide (e.g., fungicides, herbicides, insecticides, algicides, molluscicides, miticides and rodenticides) or an antimicrobial agent (e.g., germicides, antibiotics, antibacterials, antivirals, antifungals, antiprotozoals and antiparasites).

The terms “biofilm” and “microbial biofilm” refer to any group of microorganisms in which cells stick to each other on a surface. These adherent cells are frequently embedded within a self-produced matrix of extracellular polymeric substance (EPS). As used herein, “microbial biofilm” may also refer to and/or include a group of viral particles.

The terms “extracellular polymeric substances” and “EPS” refer to a generally sticky rigid structure of polysaccharides, DNA, and other organic contaminants that are produced and embedded on the surface of a microbial biofilm. A biofilm layer is anchored firmly to a surface and provides a protective environment in which microorganisms grow. Bacteria, viruses, yeasts, molds, and fungi contained in the biofilms can become dormant and therefore reduce their uptake of nutrients and/or antimicrobial agents.

The term “decontamination” refers to the neutralization or removal of dangerous substances from an area, object, surface, person, or animal.

The term “pharmacologically acceptable” as used herein to refer to, e.g., a biocide or carrier, means a chemical, compound, material, diluent, or vehicle that can be applied to surfaces, equipment, living tissue, etc. without causing undue toxicity, irritation, or allergic reaction in humans or animals.

The term “disinfectant” refers to antimicrobial agents that are applied to non-living objects to destroy microorganisms that are living on the objects and works by destroying the cell wall of microbes or interfering with microbial metabolism. Disinfection does not necessarily kill all microorganisms, especially resistant bacterial spores, and it is typically less effective than sterilization, which is an extreme physical and/or chemical process that kills all types of life. “Disinfectants” are different from other antimicrobial agents, such as antibiotics which destroy microorganisms within the body, and antiseptics which destroy microorganisms on living tissue. “Disinfectants” are also different from biocides—the latter are intended to destroy all forms of life, not just microorganisms.

The term “sanitizer” refers to substances that simultaneously clean and disinfect.

The term “eradication” means the complete destruction of a microbe colony, as demonstrated in testing of microbes in real world settings such as biofilms, such that no further microbes are detected in testing following a period of application of at least 18 minutes.

The term “hydronium” is the common name for the aqueous cation H₃O⁺, the type of oxonium ion produced by protonation of water. It is the positive ion present when an Arrhenius acid is dissolved in water, as Arrhenius acid molecules in solution give up a proton (a positive hydrogen ion, H⁺) to the surrounding water molecules (H₂O). It is the presence of hydronium ion relative to hydroxide that determines a solution's pH. The molecules in pure water auto-dissociate into “hydronium” and hydroxide ions in the following equilibrium: 2H₂O OH⁻+H₃O⁺ In pure water, there is an equal number of hydroxide and hydronium ions, so it has a neutral pH of 7. A pH value less than 7 indicates an acidic solution, and a pH value more than 7 indicates a basic solution.

The term “hard surface” generally refers to non-textile surfaces that are solid and firm to the touch and can be made of, e.g., ceramic, glass, metal, synthetic resins, melamine, formica, and plastic.

The term “soft surface” generally refers to a surface that readily yields to touch or pressure, e.g., fabrics.

The term “porous surface” generally refers to a surface that is permeable by water, air, etc.

The terms “hydrophilic-lipophilic balance” and “HLB” when referring to a surfactant is a measure of the degree to which it is hydrophilic or lipophilic, determined by calculating values for the different regions of the molecule.

The terms “lipopolysaccharides” and “LPS” are also known as lipoglycans and endotoxin, and refer to large molecules consisting of a lipid and a polysaccharide composed of O-antigen, an outer core and an inner core joined by a covalent bond. “LPS” are found in the outer membrane of Gram-negative bacteria and elicit strong immune responses in animals.

The terms “microbe” and “microorganism” are used herein to mean any bacteria, virus, or fungus, including, but not limited to, Staphylococcus aureus, Streptococcus (e.g., S. pyogenes, S. agalacticae or S. pneumoniae), Haemophilus influenzae, Pseudomonas aeruginosa, Gardnerella vaginalis, Enterobacteriacae (e.g., Escherichia coli), Clostridium perfringens, Chlamydia trachomatis, Candida albicans, Human Immunodeficiency Virus (HIV), or Herpes Simplex Virus (HSV).

The terms “methicillin-resistant Staphylococcus aureus” and “MRSA” refer to a bacterium responsible for several difficult-to-treat infections in humans. It is also called oxacillin-resistant Staphylococcus aureus (ORSA). “MRSA” is any strain of Staphylococcus aureus that has developed, through the process of natural selection, resistance to beta-lactam antibiotics, which include the penicillins (e.g., methicillin, dicloxacillin, nafcillin, oxacillin, etc.) and the cephalosporins. Strains unable to resist these antibiotics are classified as methicillin-sensitive Staphylococcus aureus, or MSSA. The evolution of such resistance does not cause the organism to be more intrinsically virulent than strains of S. aureus that have no antibiotic resistance, but resistance does make MRSA infection more difficult to treat with standard types of antibiotics and thus more dangerous.

The term “protonation” refers to the transfer of a proton to a molecule, group, or atom, such that a coordinate bond to the proton is formed. “Protonation” is a fundamental chemical reaction and a step in many stoichiometric and catalytic processes. Some ions and molecules can undergo more than one “protonation” and are labeled polybasic or polyprotic, which is true of many biological macromolecules. “Protonation” and deprotonation occur in most acid-base reactions; they are the core of most acid-base reaction theories.

The term “sterilization” refers to any process that removes, eliminates, or kills all forms of life, including transmissible agents (such as fungi, bacteria, viruses, spore forms, etc.) present in a specified region, such as a surface, a volume of fluid, medication, or in a compound such as biological culture media. “Sterilization” can be achieved with one or more of the following: heat, chemicals, irradiation, high pressure, and filtration. “Sterilization” is distinct from disinfection, sanitization, and pasteurization in that “sterilization” kills or inactivates all forms of life.

The term “surfactant” refers to a compound that lowers the surface tension (or interfacial tension) between two liquids or between a liquid and a solid. “Surfactants” may act as detergents, wetting agents, emulsifiers, foaming agents, and dispersants.

The term “titration curve” refers to a curve in the plane whose x-coordinate is the volume of titrant added since the beginning of the titration, and whose y-coordinate is the concentration of the analyte at the corresponding stage of the titration (in an acid-base titration, the y-coordinate is usually the pH of the solution).

The term “topical,” as used herein, refers to the application of the composition to any skin or mucosal surface. “Skin surface” refers to the protective outer covering of the body of a vertebrate, generally comprising a layer of epidermal cells and a layer of dermal cells. A “mucosal surface,” as used herein, refers to a tissue lining of an organ or body cavity that secretes mucous, including, but not limited to, oral, vaginal, rectal, gastrointestinal, and nasal surfaces.

The term “topically applying” means directly laying on or spreading on any skin or mucosal tissue, e.g., by use of hands or an applicator such as a wipe, puff, roller, or spray.

The term “weak acid” refers to an acid with pH above about 2.0 and below about 7.0. All pH values herein are measured in aqueous systems at 25° C. (77° F.).

All patents, patent applications, publications, technical and/or scholarly articles, and other references cited or referred to herein are in their entirety incorporated herein by reference to the extent allowed by law, as if separately set forth herein. The discussion of those references is intended merely to summarize the assertions made therein. No admission is made that any such patents, patent applications, publications or references, or any portion thereof, are relevant, material, or prior art. The right to challenge the accuracy and pertinence of any assertion of such patents, patent applications, publications, and other references as relevant, material, or prior art is specifically reserved. Although the foregoing specification and examples fully disclose and enable the present invention, they are not intended to limit the scope of the invention, which is defined by the claims appended hereto. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

This invention springs in part from the inventor's identification of the interrelation of several specific problems associated with microbial biofilms and resistance to disinfectants and cleaners. First, as physical structures around microbes, biofilms inhibit access and thereby defend against application of treatments. Second, when contacted by a treatment solution, biofilms operate to create a layer of pH equilibrium that inhibits biochemical reactions that would disrupt tenant microbes. Third, as result of the first two factors, biofilms are virtually always successful in preserving at least small pockets of microbes after contact with biocides. Because microorganisms reproduce very rapidly, any reduction in microbial contamination will be temporary and overtaken as the population growth resumes.

To effectively solve these challenges, exemplary hyperprotonation compositions and formulations are provided that are suitable for use in disinfecting, decontaminating, sterilizing, sanitizing, or cleaning a surface on which is disposed a microbial biofilm. Such surface can be a hard surface, soft surface, or porous surface and can also be living tissue, such as human or animal skin. The exemplary hyperprotonation compositions provide a concentration of highly-effective biocide, such as the natural and non-toxic GME antimicrobial biocides, as well as an efficient delivery mechanism for delivery of the antimicrobial biocides to the microbial biofilms to enable the biocides to reach the microbial biofilm at higher concentrations thereby increasing the disruption of the microbial biofilm. In addition, by combining a surfactant and a weak acid, the hyperprotonation composition operates to create a zone of hyperprotonation in what effectively is a membrane enveloping all or part of the biofilm structure. In other words, the present compositions create an enveloping membrane around the microbial biofilms that disrupts and neutralizes their defenses, and delivers safe, natural antibacterial and anti-viral active ingredients, such as the GME antimicrobial biocides. The enveloping membrane can be described as a “hydronium engine” that osmotically or, in some embodiments, through emulsion, delivers both hydronium and GME to the microbial biomass.

In one aspect, the invention features compositions and methods that are of greater efficacy in disrupting biofilms on a surface or object to be disinfected, sanitized, cleaned, and/or decontaminated. In such aspect, the invention disclosed herein incorporates a newly discovered understanding of the relationship of pH of the composition and the dynamic pH of biofilms and microorganisms within biofilms. In particular embodiments, a hyperprotonation composition is provided that includes a surfactant, one or more emulsifying agents, a biocide (or pharmacologically acceptable biocide), and a weak acid. As one skilled in the art will appreciate, surfactants are capable of functioning as emulsifiers. However, while not intending to disclaim any particular function, suitable components for use in the present compositions are chosen and identified for a particular function, e.g., surfactant, wetting agent, emulsifier, spreading agent, detergent, dispersant, or foaming agent, despite the fact that the particular component may serve some or all of these functions. In some embodiments, one or more emulsifying agents serve as a pharmacologically acceptable carrier that permits safe application of the hyperprotonation composition to the skin surface or mucosal surface of an individual.

Once applied to contaminated surface or object (i.e., a surface or object on which is disposed a microbial biofilm), the hyperprotonation composition produces a wetting layer at the surface of the microbial biofilm to increase the delivery and efficacy of biofilm disrupting agents, such as hydronium produced at the wetting layer and the biocide component, as will be explained in more detail below.

The components and agents of hyperprotonation compositions suitable for use herein will now be explained in further detail.

### Hyperprotonation Compositions

As noted above, a surfactant is employed to achieve a wetting layer at the surface of the biofilm. This surface wetting creates the equivalent of a membrane, so that osmotic pressure continues the flow of aqueous solution through the wetting layer. In preferred embodiments, the hyperprotonation composition includes one or more cationic surfactants (e.g., saponified organic acids, synthetic detergents, or a combination thereof) having a pH equal to or greater than 7. In more preferred embodiments, the cationic surfactant has a pH of at least 9. In a most preferred embodiment, the cationic surfactant is any potassium or sodium salt soap derived from one or more organic acids. In one particular non-limiting embodiment, the cationic surfactant is potassium cocoate. A suitable concentration of the cationic surfactant in the hyperprotonation composition is between about 0.5% w/v to about 10% w/v; preferably, between about 1% w/v to about 5% w/v, e.g., about 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, or 5.0% w/v. In other embodiments, the concentration of the cationic surfactant in the hyperprotonation composition is between about 5 g/L to about 100 g/L; preferably, between about 10 g/L to about 50 g/L, e.g., 10 g/L, 11 g/L, 12 g/L, 13 g/L, 14 g/L, 15 g/L, 16 g/L, 17 g/L, 18 g/L, 19 g/L, 20 g/L, 21 g/L, 22 g/L, 23 g/L, 24 g/L, 25 g/L, 26 g/L, 27 g/L, 28 g/L, 29 g/L, 30 g/L, 31 g/L, 32 g/L, 33 g/L, 34 g/L, 35 g/L, 36 g/L, 37 g/L, 38 g/L, 39 g/L, 40 g/L, 41 g/L, 42 g/L, 43 g/L, 44 g/L, 45 g/L, 46 g/L, 47 g/L, 48 g/L, 49 g/L, or 50 g/L.

In addition to a surfactant, the hyperprotonation compositions described herein may include one or more weak acids. As one skilled in the art will appreciate, weak acids typically function in solution as buffering agents and can affect the pH of the wetting layer (e.g., maintaining a low pH of the wetting layer). Weak acid buffering agents suitable for use herein typically include organic acids having a pH between about 2 and 7. Preferably, the weak acid will have a pH less than or equal to 3.5; more preferably less than or equal to 3.0. Non-limiting exemplary weak acids include, but are not limited to, citric acid (pH of about 2.2), lactic acid (pH of about 2.4), malic acid (pH of about 2.2), tartaric acid (pH of about 2.2), salicylic acid (pH of about 2.4), ascorbic acid (pH of about 3.4), and any combination of such weak acids. A suitable concentration of the weak acid, or combination of weak acids, in the hyperprotonation composition is between about 0.2% w/v to about 20% w/v; preferably, between about 0.5% w/v to about 15% w/v, e.g., about 0.5%, 1.0%, 1.5%, 2.0%, 2.5%, 3.0%, 3.5%, 4.0%, 4.5%, 5.0%, 5.5%, 6.0%, 6.5%, 7.0%, 7.5%, 8.0%, 8.5%, 9.0%, 9.5%, 10.0%, 10.5%, 11.0%, 11.5%, 12.0%, 12.5%, 13.0%, 13.5%, 14.0%, 14.5%, or 15.0% w/v. In other embodiments, the concentration of the weak acid(s) in the hyperprotonation composition is between about 2 g/L to about 200 g/L; preferably, between about 5 g/L to about 150 g/L, e.g., 5 g/L, 10 g/L, 15 g/L, 20 g/L, 25 g/L, 30 g/L, 35 g/L, 40 g/L, 45 g/L, 50 g/L, 55 g/L, 60 g/L, 65 g/L, 70 g/L, 75 g/L, 80 g/L, 85 g/L, 90 g/L, 95 g/L, 100 g/L, 105 g/L, 110 g/L, 115 g/L, 120 g/L, 125 g/L, 130 g/L, 135 g/L, 140 g/L, 145 g/L, or 150 g/L.

Once the hyperprotonation composition is contacted to a surface or object, such as a hard surface, medical equipment, or living tissue, the surfactant will form a wetting layer. If a cationic surfactant is used, the pH of the wetting layer will be much higher than that of the weak acid. As the weak acid and surfactant mix, the pH of the wetting layer changes depending on the pH difference between the weak acid and the surfactant. As one skilled in the art would readily appreciate, in an acid-base titration, the titration curve reflects the strength of the corresponding acid and base. For a strong acid and a strong base, the curve will be relatively smooth and very steep near the equivalence point. Because of this, a small change in titrant volume near the equivalence point results in a large pH change and many indicators would be appropriate (for instance litmus, phenolphthalein or bromothymol blue). If one reagent is a weak acid or base and the other is a strong acid or base, the titration curve is irregular and the pH shifts less with small additions of titrant near the equivalence point. More complex titration curves are produced by mixing polyprotic weak acids with a strong base. For instance, if a cationic surfactant is used with a high pH, such as potassium cocoate (pH of about 10), in addition to a polyprotic weak acid, such as oxalic acid or citric acid, the weak-acid/surfactant mixture may produce an irregular titration curve, the titration curve will be irregular having more than one inflection, or titration, points. The titration point, or first titration point for polyprotic acids, can therefore be used in some embodiments to select a suitable weak acid.

It is preferable that the weak acids used in the hyperprotonation compositions of the present invention have a first titration point that is lower than the pH of the surfactant. In some embodiments, suitable weak acids will have a first titration point pH of less than about 6.0. In other embodiments, the weak acid in the topical formulation will have a first titration point pH of less than about 5.0; preferably less than about 4.0. In particular embodiments, the surfactant used in the hyperprotonation composition is a cationic surfactant having a pH that is higher than the first titration point of the weak acid. In more preferred embodiments, the cationic surfactant will have a pH that is at least 2.0 units higher than the first titration point of the weak acid; most preferably, at least 3.0 units higher.

In some embodiments, the hyperprotonation composition includes a biocide. Biocides particularly suitable for use in the hyperprotonation compositions disclosed herein include antimicrobial biocides, such as germicides, antibiotics, antibacterials, antivirals, antifungals, antiprotozoals, and antiparasites. In certain embodiments, the biocide is a glycerol monoester (GME). GMEs are particularly suitable for use as biocides since they can also function as emulsifiers, analgesics, and anti inflammatory agents in hyperprotonation compositions formulated for topical application thereby providing a therapeutic benefit in addition to acting as a microbial biocide. See, e.g., U.S. 2013/0281532; Schlievert, et al. Glycerol Monolaurate Antibacterial Activity in Broth and Biofilm Cultures, 10.1371/journal.pone.0040 350 (2012), the entire contents of each of which are incorporated by reference herein.

In preferred embodiments, the GME is glycerol linked to a C6-C22 acyl group (e.g., C(═O)C5-C21 alkyl, wherein the alkyl is branched or unbranched, saturated or unsaturated). In these embodiments, the GME suitable for use has the formula R₁OCH₂(OR₂)CH₂OR₃, wherein R₁, R₂, and R₃ can either be a hydrogen (H) or a C6 to C22 acyl group. In some embodiments, the acyl group is branched or unbranched, saturated or unsaturated. In other embodiments, the acyl group is unbranched and saturated. In preferred embodiments, the acyl group is derived from a fatty acid, e.g., caprylic acid, capric acid, lauric acid, myristic acid, palmitic acid, stearic acid, arachidic acid, or behenic acid. In particular embodiments, the GME is glycerol monocaprylate (C8), glycerol monocaprate (CIO), glycerol monolaurate (CI 2, “GML”), or glycerol monomyristate (CI 4). GMEs, including GML, have been determined by the U.S. Environmental Protection Agency to be non-toxic (see 69 FR 34937) and have been listed in the Generally Recognized as Safe (GRAS) substances by the U.S. Food and Drug Administration. Indeed, GML occurs naturally in honey and human breast milk. GML and related compounds have been previously disclosed in U.S. patent application Ser. No. 10/579,108 (filed Nov. 10, 2004) and Ser. No. 11/195,239 (filed Aug. 2, 2005), the disclosures of each of which are herein incorporated by reference in their entireties. In some embodiments, the concentration of the biocide in the hyperprotonation composition is from about 10 μg/ml to about 10,000 μg/ml. In preferred embodiments, the concentration of the biocide is at least about 0.05% w/v; more preferably, at least about 0.1% w/v; most preferably, it is at least about 0.15% w/v. In some embodiments, the concentration of the biocide in the hyperprotonation composition is at least about 10 μg/ml; preferably, it is at least about 100 μg/ml; more preferably it is at least about 500 μg/ml; most preferably, it is at least about 1,000 μg/ml. In a non-limiting exemplary embodiment, a hyperprotonation composition is provided that includes about 1,500 μg/ml biocide, e.g., GML.

In an embodiment, the hyperprotonation composition includes one or more emulsifying agents. In other embodiments, the hyperprotonation composition is formulated for topical application and comprises a pharmacologically acceptable carrier that includes one or more emulsifying agents and one or more additional agents, including, but not limited to, one or more nonaqueous oils or gels. For instance, in some embodiments, the pharmacologically acceptable carrier includes olive oil, vegetable oil, and/or petroleum jelly. Emulsifying agents suitable for use herein include, but are not limited to, sorbitan monolaurate (Polysorbate 20), sodium stearoyl lactylate, polyoxyethylene (20) sorbitan monooleate (Polysorbate 80), or any combination thereof. In some embodiments, the total concentration of emulsifying agents in the hyperprotonation composition are from about 0.2% to about 10% w/v; preferably, from about 0.5% w/v to about 5% w/v, e.g., about 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, or 5% w/v. In other embodiments, the concentration of the emulsifying agents in the hyperprotonation composition is between about 2 g/L to about 100 g/L; preferably, between about 5 g/L to about 50 g/L, e.g., 5, g/L, 6 g/L, 7 g/L, 8 g/L, 9 g/L, 10 g/L, 11 g/L, 12 g/L, 13 g/L, 14 g/L, 15 g/L, 16 g/L, 17 g/L, 18 g/L, 19 g/L, 20 g/L, 21 g/L, 22 g/L, 23 g/L, 24 g/L, 25 g/L, 26 g/L, 27 g/L, 28 g/L, 29 g/L, 30 g/L, 31 g/L, 32 g/L, 33 g/L, 34 g/L, 35 g/L, 36 g/L, 37 g/L, 38 g/L, 39 g/L, 40 g/L, 41 g/L, 42 g/L, 43 g/L, 44 g/L, 45 g/L, 46 g/L, 47 g/L, 48 g/L, 49 g/L, or 50 g/L.

Other components may be included in the compositions and formulations disclosed herein. In some embodiments, the topical formulation includes thickeners, such as synthetic polymers, fatty acids, fatty acid salts and esters, fatty alcohols, modified celluloses or modified mineral materials. In such embodiments, the thickeners can also be employed with liquid carriers to form spreadable pastes, gels, ointments, soaps, and the like, for application directly to the skin or mucosal surface of a human or animal. Examples of useful dermatological compositions which can be used to deliver the actives in the hyperprotonation compositions to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508), the content of each of which is incorporated herein by reference in their entireties.

Hyperprotonation compositions of the present invention include any combination of the components described above and in any of the above-described concentrations. When the hyperprotonation composition is applied to a hard or soft surface or object, or to living tissue, on which is disposed a microbial biofilm, the surfactant forms a membrane-like wetting layer at the surface of the microbial biofilm and maintains the osmotic pressure flow of aqueous solution through the wetting layer. In addition, hyperprotonation compositions containing surfactants with a pH that is higher than that of the weak acid and, in particular, cationic surfactants having a pH of greater than 7, produce a wetting layer with an elevated pH, such that the pH of the wetting layer exceeds the first titration point of the weak acid component. By combining a weak acid with the wetting layer in proper pH-titration point balance, the invention maintains continuous and enhanced protonation in the surfactant layer, which results in ongoing creation of hydronium at the surface of the EPS as protons are donated from the weak acid to water. It is a catalytic process. Additionally, the surfactant compounds at the wetting layer and maintaining the membrane pH levels are not consumed in the process.

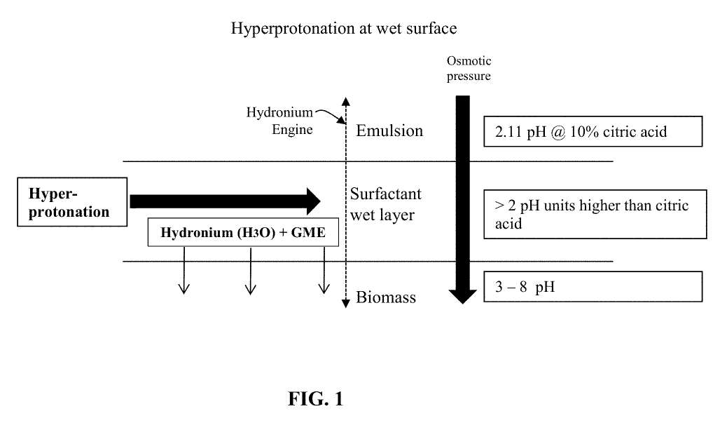

Shown in FIG. 1 is an illustration of a preferred embodiment of the wetting layer formed when the hyperprotonation composition is applied to a surface. In this non-limiting embodiment, three layers are depicted: (1) the emulsion, (2) the surfactant wetting layer, and (3) the microbial biomass. As shown in FIG. 1, the wetting layer has a pH greater than 4.11 and therefore above the lowest titration point of the citric acid disposed in the emulsion, causing titration and hyperprotonation through the wetting layer. Further, the titration event in the wetting layer does not consume the surfactant and therefore does not reach equilibrium, as would occur if there was direct contact with the biomass. The three-layer structure produced by the topical formulations described herein can be described as a “hydronium engine” as the hyperprotonation of water from acid in the wetting layer increases the hydronium available for delivery to the microbial biomass. Further, the hydronium delivery and the osmotic gradient across the layer gives the wetting layer characteristics similar to semipermeable membranes.

The hyperprotonation compositions described herein have increased efficacy due, in part, to their ability to disrupt the defenses of microbial biofilms that are formed by microbes in response to many factors, including cellular recognition of specific or non-specific attachment sites on a surface, nutritional cues, or in some cases, by exposure of planktonic cells to sub-inhibitory concentrations of antibiotics. When a cell switches to the biofilm mode of growth, it undergoes a phenotypic shift in behavior in which large suites of genes are differentially regulated.

Important to the microbial biofilm's defenses are the presence of EPS and LPS molecules. LPS is the major component of the outer membrane of Gram-negative bacteria, contributing greatly to the structural integrity of the bacteria, and protecting the membrane from certain kinds of chemical attack. LPS also increases the negative charge of the cell membrane and helps stabilize the overall membrane structure. It is of crucial importance to gram-negative bacteria, whose death results if it is mutated or removed. LPS induces a strong response from normal animal immune systems and has also been implicated in non-pathogenic aspects of bacterial ecology, including surface adhesion, bacteriophage sensitivity, and interactions with predators such as amoebae. EPS are high-molecular weight compounds secreted by microorganisms into their environment. EPS establish the functional and structural integrity of biofilms, and are considered the fundamental component that determines the physiochemical properties of a biofilm. EPS are mostly composed of polysaccharides (exopolysaccharides) and proteins, but include other macro-molecules such as DNA, lipids, and humic substances.

One of the benefits of the present hyperprotonation compositions is that they enhance protonation at the microbial biofilm surface, which disrupts the LPS and EPS defenses. Protonation is the addition of a proton to an atom, molecule, or ion. The proton is the nucleus of the hydrogen atom, and the positive hydrogen ion, H+, consists of a single proton. An example of protonation is the formation of the ammonium group NH₄+ from ammonia, NH₃. Protonation often occurs in the reaction of an acid with a base to form a salt. Protonation differs from hydrogenation in that during protonation a change in charge of the protonated species occurs, whereas the charge is unaffected during hydrogenation. Protonations are often rapid, in part because of the high mobility of protons in water. The rate of protonation is related to the acidity of the protonating species, in that protonation by weak acids is slower than protonation of the same base by strong acids. The rates of protonation and deprotonation can be especially slow when protonation induces significant structural changes.

The composition of the hyperprotonation composition effectively augments or hyper-charges the ongoing impact of the protonation by the weak acid—what is defined by this application as “hyperprotonation.” In hyperprotonation, the pH in the wetting layer remains above the titration point of the acid and thus maintains ongoing production of hydronium (heavy water H₃O) in a protonation process. By providing compositions that maintain the pH at the biofilm layer above the first titration point of the weak acid within the composition, the invention enables protonation to continue to occur, such that the microbial biofilm's EPS and LPS defenses are effectively breached. Importantly, the lower pH on the target surface is not an impediment to ongoing protonation which occurs in the wetting layer.

Another key aspect of microbial biofilm defenses is their ability to establish a pH equilibrium at the surface layer that effectively block lower pH solutions from reaching the biomass. Disrupting these defenses through hyperprotonation reduces the pH in the microbial biofilm, thereby increasing the potency of a microbial biocide to kill microbes by as much as eight orders of magnitude. See, e.g., Glycerol Monolaurate and Biofilm Technical Paper, U.S. National Institutes of Health (2012), the content of which is incorporated herein by reference in its entirety.

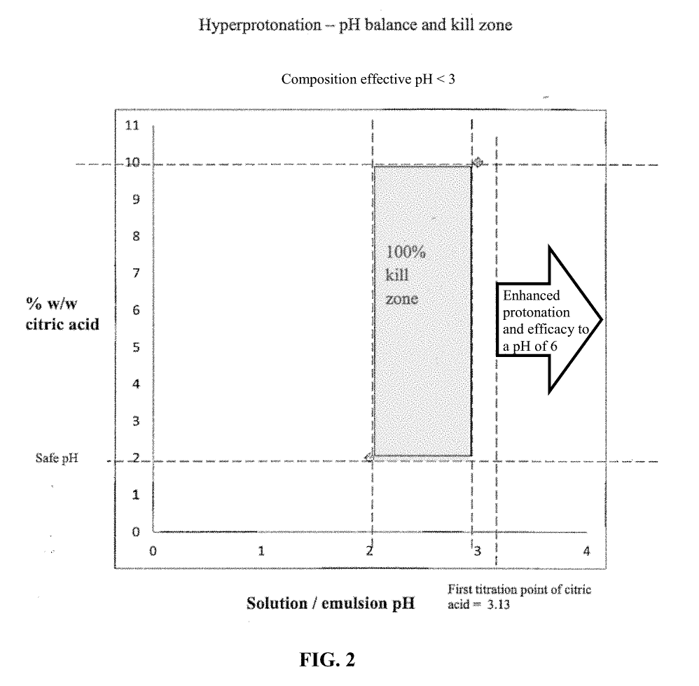

Shown in FIG. 2 is a depiction of the kill zone of an exemplary hyperprotonation composition. In FIG. 2, the biocide (e.g., GME) concentration is greater than 500 μg/ml, the surfactant concentration is greater than about 0.5% w/v, the steady state pH of the solution is not greater than the titration point of the acid, and the pH of the surfactant mix (with emulsifier and GME) is at least 2 pH units higher than the titration point of the acid.

The hyperprotonation compositions provided herein can be used for sterilization, disinfection, sanitization, and/or cleaning of any surface or object contaminated with microbes and/or microbial biofilms. Contaminated surfaces include hard surfaces and soft surfaces, such as those found in household environments, industrial environments, and also include the surfaces of food products. In addition, the hyperprotonation compositions can be used to eradicate and disrupt microbial biofilms internal or external to living tissue, such as human or animal skin or mucosa. In one embodiment, the hyperprotonation composition is formulated as a liquid formulation. In other embodiments, the hyperprotonation composition is formulated as a fog, gel, cream, spray, mist, or ointment.

### Methods of Use

The hyperprotonation compositions provided herein can be applied to any surface or object on which is disposed microorganisms and/or a microbial biofilm, as microorganisms are the cause of many infectious diseases. Indeed, these microorganisms include pathogenic bacteria that cause diseases such as plague, tuberculosis, and anthrax; protozoa that cause diseases such as malaria, sleeping sickness, dysentery, and toxoplasmosis; and fungi that cause diseases such as ringworm, candidiasis, or histoplasmosis. Other diseases such as influenza, yellow fever, or AIDS are caused by pathogenic viruses, which are not usually classified as living organisms, but, for the purposes of this disclosure, are encompassed by the microbial biofilms of the present methods.

Microbial biofilms provide a protective environment in which many of these bacteria, viruses, yeasts, molds, and fungi grow, which can become dormant within these biofilms enabling the reduction of their uptake of antimicrobial agents. These microbial biofilms have therefore been found to be involved in a wide variety of microbial infection in humans and animals, such as urinary tract infections, catheter infections, middle-ear infections, formation of dental plaque, gingivitis, coating contact lenses, and serious and potentially lethal processes such as endocarditis, infections in cystic fibrosis, and infections of permanent indwelling devices such as joint prostheses and heart valves. Microbial biofilms may impair cutaneous wound healing and reduce topical antibacterial efficiency in healing or treating infected skin wounds. Moreover, microbial biofilms are present on the removed tissue of 80% of patients undergoing surgery for chronic sinusitis and can also be formed on the inert surfaces of implanted devices such as catheters, prosthetic cardiac valves and intrauterine devices. For instance, MRSA is especially troublesome in hospitals, prisons, and nursing homes, where patients with open wounds, invasive devices, and weakened immune systems are at greater risk of nosocomial infection than the general public. MRSA began as a hospital-acquired infection, but has developed limited endemic status and is now sometimes community-acquired. The terms HA-MRSA (healthcare-associated MRSA) and CA-MRSA (community-associated MRSA) reflect this distinction.

The hyperprotonation compositions of the present invention can be applied anywhere where bacteria, viruses, yeast, and molds exist and/or where they form or are incorporated into microbial biofilms. Thus, in one embodiment, the hyperprotonation compositions of the present invention can be used to clean, disinfect, decontaminate, sterilize, or sanitize any surface, such as a hard surface, soft surface, or porous surface; piece of equipment; living tissue, such as human or animal skin, human or animal mucous membranes, or plants; or fabric, such as carpet, cloth, linen, and silk. In some embodiments, the hyperprotonation composition is applied to hard surfaces, such as countertops, walls, doors, toilets, shower stalls, bathtubs, sinks, and chairs typically found in households or office buildings. In other embodiments, the hyperprotonation composition is applied to the interior and/or exterior of equipment used in the food, scientific, and medical industries. The hyperprotonation compositions can also be used for the cleaning, disinfection, and/or sterilization of sports or fitness facilities, including, but not limited to, lockers, locker rooms, gymnasium floors and bleachers, showers, and bathrooms.

In some aspects, the hyperprotonation composition is applied to the surface of food products, such as fruits, vegetables, and meat. In other aspects, the hyperprotonation composition is applied to the interior and/or exterior surfaces of a human or animal body (e.g., skin surface or mucosal surface). In other embodiments, the hyperprotonation compositions can be used as a skin wash, surgical wash, carcass wash, or as an initial step in a sterilization sequence for medical devices.

In addition, the hyperprotonation compositions provided herein can be incorporated into other cleaning compositions, such as toilet bowl cleaners, metal cleaners and brighteners, rust stain removers, denture cleansers, metal descalers, general hard surface cleaners, and disinfectants, thus providing these cleaners with additional enhanced microbial biofilm disruption capability.

In other embodiments, the hyperprotonation composition is applied to any surface for the control and/or eradication of gram positive bacteria, gram negative bacteria, viruses, yeasts, and molds existing in or incorporated into microbial biofilms.

The hyperprotonation compositions provided herein can be applied directly to any surface as a gel, cream, liquid, mist, fog, ointment, soak, or spray. Further, hyperprotonation composition can be applied to surfaces through flood application, spray application, high-pressure application, foam application, or clean-in-place application.

Once applied to a surface, the hyperprotonation compositions of the present invention can be left on the treatment area for a period of about 30 seconds or more, e.g., 30 sec., 40 sec., 50 sec., or more, prior to removing the hyperprotonation composition from the treatment area (e.g., by rinsing or washing). In other embodiments, the hyperprotonation compositions are left on the treatment area for a period of at least about 1 min., e.g., 1, 2, 3, 4, 5, 6, 7, 8, 9, 10, 11, 12, 13, 14, 15, 16, 17, 18, 19, 20 min., or more. In yet other embodiments, the hyperprotonation compositions are left on the treatment area for about 1 hour or more, e.g., 1, 2, 3, 4, 5, 6, 7, 8, 9, 10 hours, or more.

Moreover, many of the exemplary compositions and methods disclosed herein have the further benefit of being generally regarded as safe (GRAS) by the U.S. FDA for use on food and/or are acceptable under the regulations of the USDA National Organic Production (NOP) and are completely biodegradable.

A person skilled in the art would recognize that the compositions disclosed herein can be made in concentrated form and then diluted to achieve proportions of acids as above. A benefit of the invention is that it operates effectively on a broad spectrum basis. It can reliably eradicate both gram-positive and gram-negative microorganisms, as well as combinations of microorganisms where the precise chemical composition is indeterminate.

The following examples describe the invention in greater detail. They are intended to illustrate, rather than to limit, the invention.

### Examples

### Example 1. Exemplary Hyperprotonation Composition Formulation

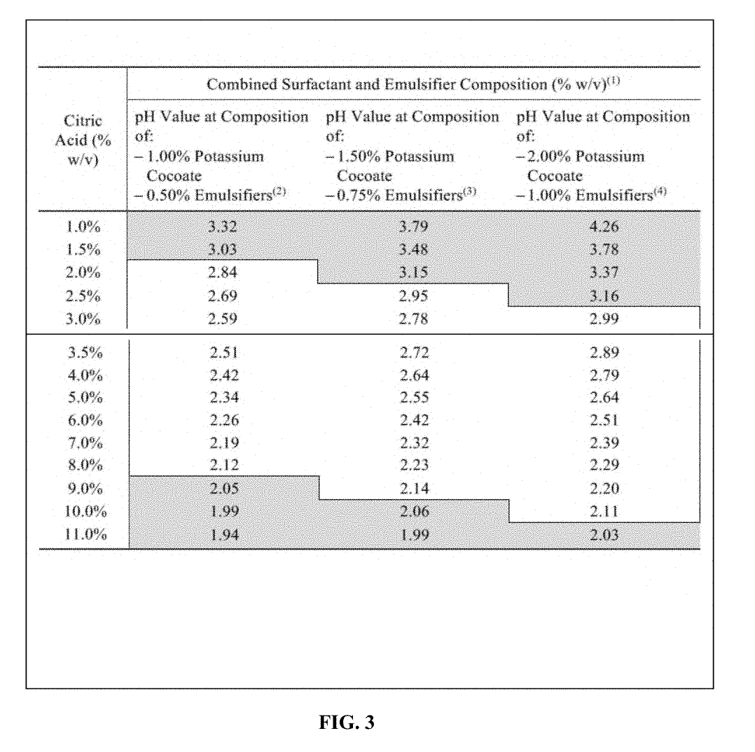

Mixing stable emulsion compositions are well within the purview of the skilled artisan and will not be discussed in detail herein. A non-limiting exemplary hyperprotonation composition was produced having the components described in Table 1. Potassium cocoate was chosen as the cationic surfactant, and GML was chosen as the biocide. Furthermore, emulsifying agents (i.e., sorbitan monolaurate and sodium stearoyl lactylate) were added. The concentration of citric acid was chosen for this particular composition based upon how different concentrations of citric acid affect the pH of the potassium cocoate and emulsifier mixture (see FIG. 3).

TABLE 1
Exemplary Hyperprotonation Formulation

| Component | CAS* Registry No. | % w/v | g/L |
|---|---|---|---|
| Water | | 87.00% | 870.00 |
| Potassium Cocoate | 61789-30-8 | 2.00% | 20.00 |
| Sorbitan Monolaurate | 9005-64-5 | 0.80% | 8.00 |
| (Polysorbate 20) | | | |
| Sodium Stearoyl | 25383-99-7 | 0.05% | 0.50 |
| Lactylate | | | |
| GML | 142-18-7 | 0.15% | 1.50 |
| Citric Acid | 77-92-9 | 10.00% | 100.00 |
| Total | | 100% | 1000.00 |

*CAS, Chemical Abstracts Service.

### Example 2. Antimicrobial Performance Testing

An exemplary hyperprotonation composition as described in Table 1 was tested under the conditions described for hospital grade disinfectant according to Schedule 1 of the Therapeutic Goods Order No. 54 and as described in Kelsey and Maurer, Pharm. J. 213:528-530 (1978), the entire content of which is incorporated herein by reference. The hyperprotonation composition was tested neat (with no dilution) and was challenged with bacterial inoculum followed by sampling of this mix at a prescribed time point, rechallenged with the same hyperprotonation composition vial, and sampled again at a later prescribed time point. The sample was cultured in a suitable recovery medium for 48 hr. The organisms used were:

Escherichia coli NCTC 8196;

Pseudomonas aeruginosa NCTC 6749;

Staphylococcus aureus NCTC 4163;

Proteus vulgaris NCTC 4635; and

Listeria monocytogenes A19115.

The hyperprotonation composition was tested with each of these organisms under both ‘clean’ and ‘dirty’ conditions. Clean conditions consisted of resuspension of the test organism in sterile hard water. Dirty conditions consisted of resuspension of the test organism in a sterile yeast suspension (which acted as an organic soil). The hyperprotonation composition passed or failed the assay according to the extent of growth in each of 5 recovery broth tubes at each time point in an assay that was considered valid ie., 10 test vials in total. Validity of the assay depended on the number of organisms/ml in the starting inoculum, which was measured at the time of the assay, and that the expected results were obtained for each of 4 controls. These controls ensured the sterility of the recovery medium, the sterility of the formulation, the growth of the organism and that the hyperprotonation composition sample was sufficiently inactivated when the sample was added to the recovery medium and therefore allowed the organism to grow if it had not been killed during incubation with the hyperprotonation composition.

For testing, each of the control organisms were required to have been subcultured at least 5, but not more than 14 times (i.e., days in a row). The hyperprotonation composition was required to be tested with each organism under clean and dirty conditions in 3 valid assays carried out over subsequent days.

For E. coli, P. aeruginosa, S. aureus, and P. vulgaris, the contents of an ampoule of freeze-dried culture was incubated overnight at 37° C.+/−1° C. in Wright and Mundy dextrose medium. The incubated culture was inoculated onto nutrient agar slopes in McCartney bottles and stored for up to 3 months at 4° C.+/−1° C. Prior to the test, the culture was subcultured from the agar slope into 10 ml or 15 ml quantities of Wright and Mundy dextrose medium and incubated at 37° C.+/−1° C. for 24+/−2 hours. The subculture was subcultured a second time into fresh medium, using an inoculating loop of about 4 mm in diameter and incubated at 37° C.+/−1° C. for 24+/−2 hours. This step was repeated daily until testing was performed. For the test procedure only those cultures which have been subcultured at least 5, but not more than 14 times, were used.

For L. monocytogenes, a bead from a glycerol stock was inoculated on an HBA plate and incubated overnight at 37° C.+/−1° C. The incubated culture was inoculated onto nutrient agar slopes in McCartney bottles and stored for up to 3 months at 4° C.+/−1° C. Prior to the test, the culture was sub-cultured from the agar slope into 10 ml or 15 ml quantities of BHI medium and incubated at 37° C.+/−1° C. for 24+/−2 hours. The subculture was subcultured a second time into fresh medium, using an inoculating loop of about 4 mm in diameter and incubated at 37° C.+/−1° C. for 24+/−2 hours. This step was repeated daily until testing was performed. For the test procedure only those cultures which have been subcultured at least 5, but not more than 14 times, were used.

Prior to centrifugation, test cultures of P. aeruginosa and S. aureus were filtered through sterile Whatmans No. 4 filter paper. All test cultures were then centrifuged until the cells were compact. Then, the supernatant was removed with a Pasteur pipette, and the test organisms were resuspended in the original volume of liquid (i.e., 10 ml or 15 ml) and shaken for 1 minute with a few sterile glass beads. For the “clean” assay conditions, the test organisms were resuspended in sterile hard water. For the “dirty” assay conditions, the test organisms were resuspended in a mixture of 4 parts yeast suspension to 6 parts sterile hard water.

Immediately before testing, the resuspended inoculums were sampled and enumerated using 10-fold dilutions in quarter-strength Ringer's solution and the pour-plate technique. The number subsequently counted was required to represent not less than 2×10⁸ or more than 2×10⁹ organisms per ml or the test was considered invalid. A tube containing the 10⁻⁷ dilution was used for the controls.

Samples of the hyperprotonation composition was quantitatively diluted to the specified extent, using sterile hard water as diluent. No less than about 10 ml or about 10 g of each sample was used for the first dilution, and no less than 1 ml of any dilution was used to prepare any subsequent dilutions. All dilutions were done in glass containers on the day of testing. The glass containers were twice rinsed in glass-distilled water, and sterilized. Containers were tested at a controlled temperature of 21° C.+/−1° C. either by maintaining the testing environment at this temperature or by use of a water bath.

Next, hyperprotonation composition samples for testing were prepared by adding 3 ml of diluted hyperprotonation composition sample to a capped glass container and immediately inoculating with 1 ml of test culture and mixing by swirling. At 8 minutes, one drop (0.02 ml+/−0.002 ml) of each sample was subcultured into each of 5 tubes containing recovery broth. At 10 minutes, each hyperprotonation composition sample was inoculated a second time with 1 ml of test culture and mixed by vortexing. At 18 minutes, one drop (0.02 ml+/−0.002 ml) of each hyperprotonation composition sample was subcultured into each of 5 tubes containing recovery broth. All tubes of recovery broth were mixed by vortexing and incubated at 37° C.+/−1° C. for 48+/−2 hours. Next, each tube of recovery broth was examined for growth, and the results were recorded. For each test organism, the test procedure was repeated on each of 2 subsequent days using a fresh hyperprotonation composition sample and a freshly prepared bacterial suspension.

For the recovery broth contamination control, 1 uninoculated tube of recovery broth was incubated at 37° C.+/−1° C. for 48+/−2 hours and examined for growth. If growth occurred, the test was considered invalid due to contamination of the recovery broth. For the hyperprotonation composition contamination control, 0.02 ml of diluted hyperprotonation composition sample was added to 1 tube of recovery broth and incubated at 37° C.+/−1° C. for 48+/−2 hours and examined for growth. If growth occurred, the test was considered invalid due to contamination of the hyperprotonation composition test sample. To ensure that the test organisms were viable, 1 ml of the 10⁻⁷ microbial dilution obtained above was added to 1 tube of recovery broth and incubated at 37° C.+/−1° C. for 48+/−2 hours and examined for growth. If no growth occurred, the test was considered invalid. To determine the inactivator efficacy, 2 ml of diluted hyperprotonation composition was added to 1 ml of the 10⁻⁷ microbial dilution obtained above and incubated at 37° C.+/−1° C. for 48+/−2 hours and examined for growth. If growth occurred in the organism viability control, but no growth occurred in the hyperprotonation composition/microbial tube, the test was considered invalid due to inadequate inactivation of the hyperprotonation composition sample. Any invalid test was repeated.

The dilution test passed if there was no apparent growth in at least two out of the five recovery broths in the 8 minute sampling and no apparent growth in at least two of the five recovery broths in the 18 minute sample on all three occasions with all four organisms. As shown in Table 3, the exemplary hyperprotonation composition passed every assay with each test organism under both clean and dirty conditions. For E. coli, P. aeruginosa, S. aureus, and P. vulgaris, no growth was shown in any of the recovery tubes.

TABLE 3
Performance Results.

| | | | | Positive | Positive |
|---|---|---|---|---|---|
| | | | | Cultures | Cultures |
| | Assay | Assay | Assay | at 8 | at 18 |
| Organism | Conditions | Number | Validity | min | min |
| E. coli | Clean | 1 | Valid | 0 | 0 |
| | | 2 | Valid | 0 | 0 |
| | | 3 | Valid | 0 | 0 |
| | | 4 | Valid | 0 | 0 |
| | Dirty | 1 | Valid | 0 | 0 |
| | | 2 | Invalid | 0 | 0 |
| | | 3 | Valid | 0 | 0 |
| | | 4 | Valid | 0 | 0 |
| P. aeruginosa | Clean | 1 | Valid | 0 | 0 |
| | | 2 | Invalid | 0 | 0 |
| | | 3 | Valid | 0 | 0 |
| | | 4 | Valid | 0 | 0 |
| | Dirty | 1 | Valid | 0 | 0 |
| | | 2 | Valid | 0 | 0 |
| | | 3 | Valid | 0 | 0 |
| S. aureus | Clean | 1 | Valid | 0 | 0 |
| | | 2 | Valid | 0 | 0 |
| | | 3 | Valid | 0 | 0 |
| | Dirty | 1 | Valid | 0 | 0 |
| | | 2 | Valid | 0 | 0 |
| | | 3 | Valid | 0 | 0 |
| P. vulgaris | Clean | 1 | Valid | 0 | 0 |
| | | 2 | Valid | 0 | 0 |
| | | 3 | Valid | 0 | 0 |
| | Dirty | 1 | Invalid | 0 | 0 |
| | | 2 | Valid | 0 | 0 |
| | | 3 | Valid | 0 | 0 |
| | | 4 | Valid | 0 | 0 |
| L. monocytogenes | Clean | 1 | Valid | 0 | 0 |
| | | 2 | Valid | 0 | 0 |
| | | 3 | Valid | 0 | 0 |
| | Dirty | 1 | Valid | 2 | 0 |
| | | 2 | Valid | 1 | 0 |
| | | 3 | Valid | 1 | 0 |

A separate batch of hyperprotonation composition was evaluated in triplicate using the same test protocol described above. Shown in Table 4 are the results for the “clean” assay, whereas the results in Table 5 represent the “dirty” assay.

TABLE 4
Clean Assay Results.

| | | Count | Growth in Recovery Broths | | |
|---|---|---|---|---|---|
| Test | Dilution (v/v) | (Orgs/ml) | Challenge 1 | Challenge 2 | Results |

Escherichia coli NCTC 8196

| | | | | | |
|---|---|---|---|---|---|
| 1 | Neat | 1.4 × 10⁹ | — | — | Pass |
| 2 | Neat | 9.5 × 10⁸ | — | — | Pass |
| 3 | Neat | 4.3 × 10⁸ | — | — | Pass |

Proteus vulgaris NCTC 4635

| | | | | | |
|---|---|---|---|---|---|
| 1 | Neat | 6.5 × 10⁸ | — | — | Pass |
| 2 | Neat | 8.5 × 10⁸ | — | — | Pass |
| 3 | Neat | 5.1 × 10⁸ | — | — | Pass |

Pseudomonas aeruginosa NCTC 6749

| | | | | | |
|---|---|---|---|---|---|
| 1 | Neat | 3.2 × 10⁸ | — | — | Pass |
| 2 | Neat | 5.6 × 10⁸ | — | — | Pass |
| 3 | Neat | 5.8 × 10⁸ | — | — | Pass |

Staphylococcus aureus NCTC 4163

| | | | | | |
|---|---|---|---|---|---|
| 1 | Neat | 2.5 × 10⁸ | — | — | Pass |
| 2 | Neat | 2.5 × 10⁸ | — | — | Pass |
| 3 | Neat | 3.1 × 10⁸ | — | — | Pass |

TABLE 5
Dirty Assay Results.

| | | Count | Growth in Recovery Broths | | |
|---|---|---|---|---|---|
| Test | Dilution (v/v) | (Orgs/ml) | Challenge 1 | Challenge 2 | Results |

Escherichia coli NCTC 8196

| | | | | | |
|---|---|---|---|---|---|
| 1 | Neat | 8.3 × 10⁸ | — | — | Pass |
| 2 | Neat | 8.0 × 10⁸ | — | — | Pass |
| 3 | Neat | 8.8 × 10⁸ | — | — | Pass |

Proteus vulgaris NCTC 4635

| | | | | | |
|---|---|---|---|---|---|
| 1 | Neat | 1.2 × 10⁸ | — | — | Pass |
| 2 | Neat | 2.8 × 10⁸ | — | — | Pass |
| 3 | Neat | 4.7 × 10⁸ | — | — | Pass |

Pseudomonas aeruginosa NCTC 6749

| | | | | | |
|---|---|---|---|---|---|
| 1 | Neat | 1.2 × 10⁹ | — | — | Pass |
| 2 | Neat | 6.5 × 10⁸ | — | — | Pass |
| 3 | Neat | 1.4 × 10⁹ | — | — | Pass |

Staphylococcus aureus NCTC 4163

| | | | | | |
|---|---|---|---|---|---|
| 1 | Neat | 1.3 × 10⁹ | — | — | Pass |
| 2 | Neat | 7.8 × 10⁸ | — | — | Pass |
| 3 | Neat | 4.2 × 10⁸ | — | — | Pass |

The exemplary hyperprotonation composition was further evaluated using the AOAC Hard Surface Carrier Test 991.47,48,49 using undiluted samples. Briefly, the undiluted hyperprotonation composition samples were contacted for 10 minutes with the following test organisms in 5% horse serum:

Pseudomonas aeruginosa ATCC 15442;
Staphylococcus aureus ATCC 6538; and
Salmonella choleraesuis ATCC 10708.

As shown in Table 6, there were only 2 positive carriers for each of the P. aeruginosa and S. aureus samples, whereas the hyperprotonation composition eliminated S. choleraesuis in all of the carriers tested.

TABLE 6
Hard Surface Carrier Results.

| | No. of | No. of | No. of |
|---|---|---|---|
| | Carriers | Carriers | Carriers |
| Test Organism | Tested | Negative | Positive |
| Pseudomonas aeruginosa | 60 | 58 | 2 |
| Staphylococcus aureus | 60 | 58 | 2 |
| Salmonella choleraesuis | 60 | 60 | 0 |

The exemplary hyperprotonation composition was further evaluated using the BS EN 1276:2009 using 80% v/v diluted samples. Briefly, the hyperprotonation composition samples were contacted for 2, 5, or 10 minutes with Vancomycin resistant Enterococcus faecium or Methicillin resistant Staphylococcus aureus in 0.3% bovine albumin (dirty assay) at 20° C. The results are shown in Table 7.

TABLE 7
Antibiotic Resistant Bacteria Evaluation Results.

| | Initial | | | | | | |
|---|---|---|---|---|---|---|---|
| | Counter | Final Count per mL | | | Log Reduction | | |
| Organism | per mL | 2 min. | 5 min. | 10 min. | 2 min. | 5 min. | 10 min. |
| Vancomycin resistant | 8.1 × 10⁷ | <10 | <10 | <10 | >5.0 | >5.0 | >5.0 |
| Enterococcus faecium | | | | | | | |
| Methicillin resistant | 6.4 × 10⁷ | 1.0 × 10⁵ | 8.0 × 10¹ | <10 | 2.8 | >5.0 | >5.0 |
| Staphylococcus aureus | | | | | | | |

### Example 3. Evaluation of Exemplary Formulations on Microbial Biofilms

A microbial challenge study was performed using microbial biofilms to determine the antimicrobial efficacy of an exemplary hyperprotonation composition with contact times of 30 sec., 1 min., 5 min., and 10 min. against artificially produced biofilms derived from Escherichia coli, Staphylococcus aureus, and Salmonella ssp. Testing was performed in a standard microbiological laboratory employing standard techniques for handling BSL2 microorganisms. Standard PPE and facility notifications per MMDG procedures were followed. Biofilms were developed on borosilicate glass coupons (disks).

A sterile swab of each challenge organism was aseptically taken from stock cultures maintained at 2-8° C. and aseptically transferred to sterile TSA slants. The fresh slants were incubated at 30-35° C. for 18-24 hours. Ten (10) ml of TS saline was pipetted into each slant subsequent to incubation and the growth mechanically dislodged with a sterile cotton-tipped applicator. The suspension was transferred to a sterile 50 ml polypropylene centrifuge tube and washed by centrifugation at 4,000×g for 8-10 min. The supernatant was then decanted and the pellet suspended in 10 ml of saline TS. The suspension was washed a second time and suspended in 10 ml of saline TS. The organism concentration was adjusted to about 10⁸ colony forming units (cfu)/mL based on MMDG historical % T₆₂₀ nm spectrophotometer values.

Disks were wiped with sterile 70% IPA to ensure that no residual oils remained on their surface following handling. The CDC bioreactor was filled to its working volume with 300 mg/L TSB and sterilized in a standard 20-minute liquid steam cycle. The bioreactor was allowed to cool to room temperature. Next, nutritive growth medium (TSB) was prepared at 100 mg/L and sterilized. The bioreactor was acclimated to room temperature. Using sterile tubing, the bioreactor was attached to the source of growth medium. A peristaltic pump was placed between the reactor and the media source to modulate the flow rate. Waste was collected in a separate vessel. Sixteen (16) disks were placed into the reactor representing controls and 12 test surfaces (4 each) for each of 3 antimicrobial challenges. The bioreactor was seeded with one 1 ml of the challenge organism and, operated statically (batch phase) for 24+/−8 hours. The peristaltic pump was turned on following the static operation and the reactor was run in continuous flow mode for an additional 24+/−8 hours at room temperature.

Each disk was removed from the reactor and rinsed gently with sterile TS Saline to remove loosely adhered and planktonic cells and then placed individually into sterile glass beakers containing 10 ml of the test article. The disks were allowed to incubate in the test hyperprotonation composition at ambient temperature for 30 seconds, 1 min., 5 min., and 10 min. Following exposure to the test article, disks were removed from their respective beakers and placed into 10 ml of sterile DEB in a glass test tube to neutralize the test hyperprotonation composition and stop the reaction.

The organisms were removed from the test surfaces and controls through sonication for 20 minutes at room temperature followed by thorough mixing. Serial dilutions of the recovered organisms were performed; 1.0 ml samples of the serial dilutions were plated in duplicate and overpoured with sterile TSA. Plates were incubated under aerobic conditions at 30-35° C. for 3 to 5 days and the recovered organisms quantified.

The log number of microorganisms on the non-treated (no exposure to the test formulation) materials and that of the corresponding materials exposed to the test hyperprotonation composition indicates the reduction in log units.

Log reduction unit=Log A−Log B
Log A=the log number of microorganisms harvested from the non-treated control materials.
Log B=the log number of microorganisms harvested from the corresponding materials exposed to the test hyperprotonation composition.

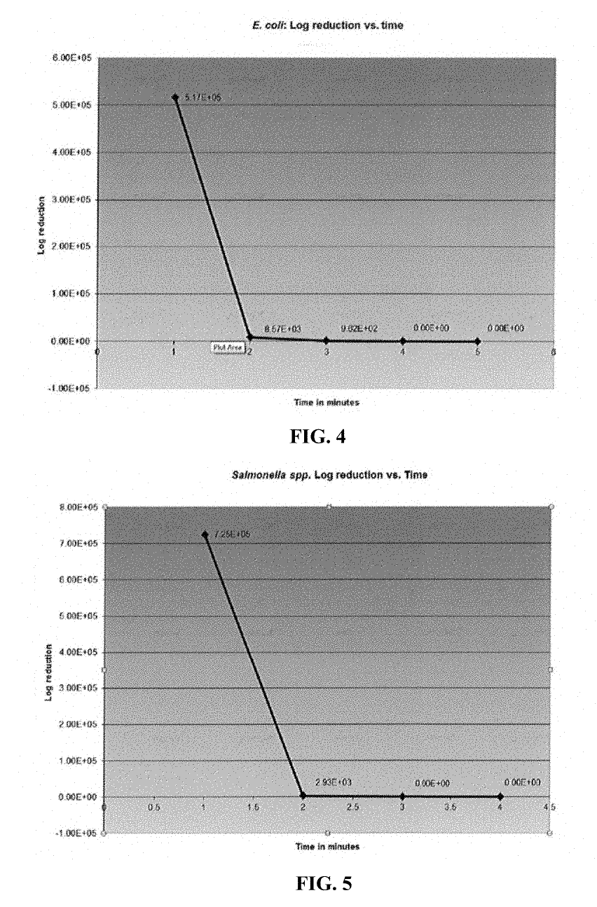

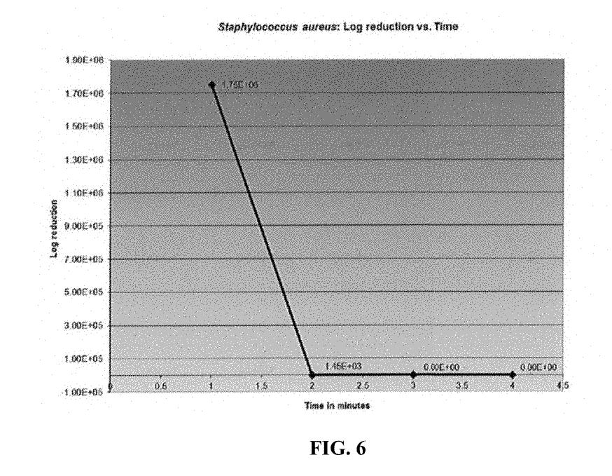

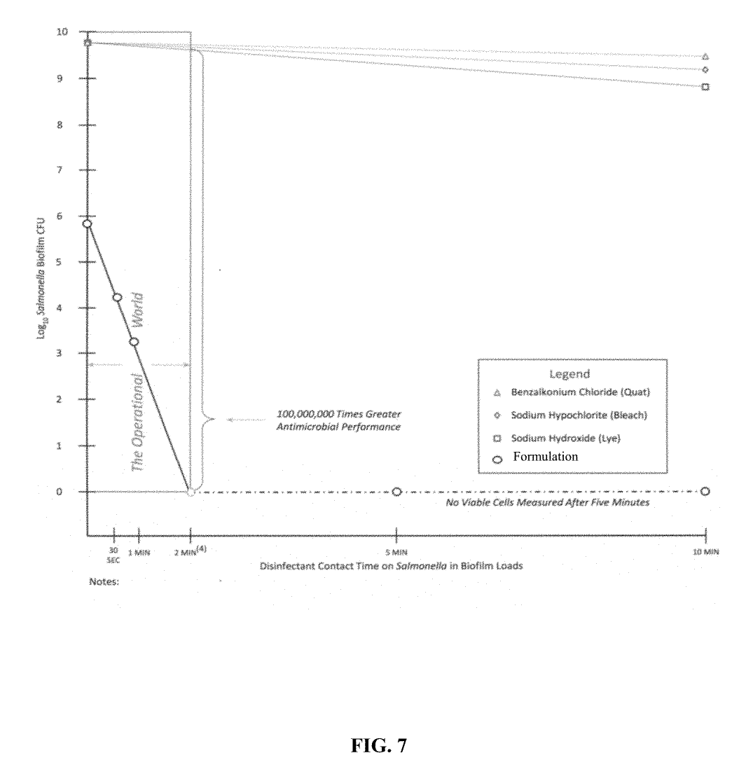

A recovery medium control was performed by first diluting the test hyperprotonation composition 1:10 in DEB and compared to a control sample of 10 ml TSB. Both the DEB and TSB samples were inoculated with about 100 cfu of the challenge organism and 1 ml samples were plated in duplicate. The recovery in the neutralized medium was compared to that of the TSB control. The recovery control results are shown in Table 8, and reveal that the recovery of the microbial challenge for all three organisms was greater than 50%. The results of the microbial biofilm challenge study is shown in Tables 9-12 and FIGS. 4-6. FIG. 7 shows the performance of the hyperprotonation composition as compared to other commercial antibacterial disinfectants.

TABLE 8
Recovery Control Results.
Recovery Medium Control

| | | | | | Neu- | | | | % |
|---|---|---|---|---|---|---|---|---|---|
| Organism | Control | CFU | | Ave | tralizer | CFU | | Ave | Recovery |
| E. coli | TSB | 122 | 147 | 135 | DEB | 109 | 128 | 119 | 88 |
| Salmonella | TSB | 78 | 86 | 82 | DEB | 66 | 70 | 68 | 83 |
| S. aureus | TSB | 39 | 46 | 43 | DEB | 34 | 50 | 42 | 99 |

TABLE 9
E. coli Challenge Results.

| | | CFU recovered | | CFU recovered | | CFU recovered | | | Average × |
|---|---|---|---|---|---|---|---|---|---|
| Sample | Dilution | #1 | | #2 | | #3 | | Average | Dilution |
| Control | 1 × 10⁴ | 51 | 46 | 33 | 69 | 48 | 63 | 52 | 5.17 × 10⁵ |
| 30 sec. | 1 × 10² | 79 | 77 | 103 | 94 | 88 | 73 | 86 | 8.57 × 10³ |
| 1 min. | 1 × 10¹ | 99 | 81 | 106 | 101 | 97 | 93 | 93 | 9.62 × 10² |
| 5 min. | 1 × 10⁰ | 0 | 0 | 0 | 0 | 0 | 0 | 0 | — |
| 10 min. | 1 × 10⁰ | 0 | 0 | 0 | 0 | 0 | 0 | 0 | — |

TABLE 10
Salmonella spp. Challenge Results.

| | | CFU recovered | | CFU recovered | | CFU recovered | | | Average × |
|---|---|---|---|---|---|---|---|---|---|
| Sample | Dilution | #1 | | #2 | | #3 | | Average | Dilution |
| Control | 1 × 10⁴ | 51 | 39 | 106 | 101 | 60 | 78 | 73 | 7.25 × 10⁵ |
| 1 min. | 1 × 10¹ | 269 | 301 | 312 | 319 | 285 | 270 | 293 | 2.93 × 10² |
| 5 min. | 1 × 10⁰ | 0 | 0 | 0 | 0 | 0 | 0 | 0 | — |
| 10 min. | 1 × 10⁰ | 0 | 0 | 0 | 0 | 0 | 0 | 0 | — |

TABLE 11
S. aureus Challenge Results.

| | | CFU recovered | | CFU recovered | | CFU recovered | | | Average × |
|---|---|---|---|---|---|---|---|---|---|
| Sample | Dilution | #1 | | #2 | | #3 | | Average | Dilution |
| Control | 1 × 10⁴ | 194 | 171 | 156 | 183 | 180 | 166 | 175 | 1.75 × 10⁶ |
| 1 min. | 1 × 10¹ | 144 | 157 | 130 | 139 | 155 | 142 | 145 | 1.45 × 10³ |
| 5 min. | 1 × 10⁰ | 0 | 0 | 0 | 0 | 0 | 0 | 0 | — |
| 10 min. | 1 × 10⁰ | 0 | 0 | 0 | 0 | 0 | 0 | 0 | — |

TABLE 12
Antimicrobial Properties vs. Time.

| Time | | E. coli (CFU) | Salmonella (CFU) | Staph. (CFU) |
|---|---|---|---|---|
| 0 | | 517,000 | 725,000 | 1,750,000 |
| 30 | sec. | 8,570 | 11,900 | 28,600 |
| 1 | min. | 962 | 2,930 | 1,450 |
| 5 | min. | 0 | 0 | 0 |

### Example 4. Mold Remediation of a Residential Apartment Case Study

A three bedroom apartment required decontamination of three rooms and removal of mold affected carpet. The remediation involved manual cleaning techniques as recommended by IICRC S520/R520 (IICRC, 2015) and a final antimicrobial treatment using fogging with a liquid hyperprotonation composition as described in Table 1, except that the concentration of citric acid is 9.5% w/v instead of 10.0% w/v. The affected carpet and rooms were identified as Condition 3, that is, contaminated with the presence of actual mold (IICRC, 2015) and were treated using recommended process under the IICRC S520. This process is called a “HEPA Sandwich” which involves HEPA Vacuuming of the affected areas, then physical removal of mold biofilm using a liquid disinfectant and then a final HEPA vacuum again.

A hyperprotonation composition (9.5% w/v citric acid) was applied using Ultra Low Volume (ULV) fogging. The fogging was performed using two (2) Scintex ULV Foggers (Scintex, Eagle Farm QLD, Australia) and was applied to all surfaces and equipment within the space. The vapor droplet size varied between 2-5 microns as per manufacturers procedure. Fogging with the hyperprotonation composition was conducted in the 5 rooms of the apartment and involved fogging the ceilings, walls and floor in the rooms. The hyperprotonation composition fog solution was left for 10 minutes and then was wiped off with a cloth moving from top to bottom.

Testing for fogged areas involved Adenosine triphosphate (ATP) testing (Hygiena Corporation) which is an Infection Control and Food Science Test used to determine the effectiveness of a decontamination process. ATP is an enzyme that is present in all living cells, and an ATP test system can detect the extent of biologicals that remains after cleaning an environmental surface, a medical device or a surgical instrument. Hospitals are using ATP-based sanitation monitoring systems to detect and measure ATP on surfaces as a method of ensuring the effectiveness of their facilities' decontamination processes. The test involved taking a swab sample in a 10×10 cm area then placing the swab in prepared media and measuring the ATP levels in an ATP Photometer (Hygiena Corporation). The results are shown in Tables 8 and 9. The decontamination process obtained no high level ATP thereby confirming that the hyperprotonation composition was effective at decontaminating the residential apartment.

TABLE 8
Pass, Caution, and Fail Level Criteria

| | Pass | Caution | Fail |
|---|---|---|---|
| Easy to Clean | <100 | 101-199 | >200 |
| Hard to Clean | <100 | 101-299 | >300 |

TABLE 9
ATP Swab Test Results

| Sample | Location | ATP Level | Result |
|---|---|---|---|
| Pre-fogging | Bedroom 1 | 289 | Caution |
| Pre-fogging | Bedroom 2 | 122 | Caution |
| Pre-fogging | Bedroom 3 | 267 | Caution |
| Post-fogging | Bedroom 1 | 0 | Pass |
| Post-fogging | Bedroom 2 | 2 | Pass |
| Post-fogging | Bedroom 3 | 0 | Pass |

### REFERENCES

Barger et al., Cleaning and disinfecting compositions with electrolytic disinfecting booster, U.S. Pat. No. 6,255,270 (Jul. 3, 2001).
Bueg-Deeb, et al., Methods and compositions for cleaning and disinfecting surfaces, U.S. Pub. No. 2014/0275267 (Sep. 18, 2014).
W. G. Characklis, Microbial Biofouling Control in Biofilms, Characklis and Marshall, eds., Wiley & Sons, 1990, J. W. Costerton, Ann Rev. Microb. 49:7110-7145, 1995.
Donelli, G. ed., Biofilm-based Healthcare-associated Infections, Volume 1 (Springer 2015)
Donelli, G. ed., Biofilm-based Healthcare-associated Infections, Volume 2 (Springer 2015),
Hidalgo, et al., Functional Tomographic Fluorescence Imaging of pH Microenvironments in Microbial Biofilms by Use of Silica Nanoparticle Sensors, Appl Environ Microbiol. 2009 December; 75(23): 7426-7435
James et al., Wound Rep Reg. 16: 37-44, 2008
McCutcheon's, Detergents and Emulsifiers, North American Edition, 1991
Nusbaum et al., Biofilms in Dermatology, Skin Therapy Lett. (2012 July; 17(7))
Ruzin, et al., Equivalence of Lauric Acid and Glycerol Monolaurate as Inhibitors of Signal Transmission in Staphylococcus aureus, Journal of Bacteriology (May 2000)
Schlievert, et al., Glycerol Monolaurate Antibacterial Activity in Broth and Biofilm Cultures, PloS One (2012)
Schlievert, Compositions for topical treatment of microbial infections, U.S. Pub. No. 2013/0281532 (Oct. 24, 2013)
Silvester, Amine-acid thickening compositions, EP 0253676 (1988)
Singh and Barbul, Wound Rep Reg. 16: 1, 2008
Urban, Hard surface cleaning compositions and method of removing stains, U.S. Pat. No. 6,936,579 (Aug. 30, 2005)
Whitley et al., Biofilm remover, U.S. Pat. No. 8,012,461 (Sep. 6, 2011)
Physicochemical characterization and evaluation of a microemulsion system for antimicrobial activity of glycerol monolaurate, doi:10.1016/j.ijpharm.2006.05.019
Compositions for topical treatment of microbial infections, WO 2013159029
Inhibition of biofilm formation by 1,2,3,4,6-penta-o-galloyl-d-glucopyranose, U.S. Pub. No. 2012/0321566
COMPOSITIONS FOR TREATING BIOFILM, U.S. Pat. No. 6,830,745
A composition for use on skin and wound, WO 2010070292
CONTROL OF BIOFILM FORMATION, U.S. Pub. No. 2014/0205586
INHIBITING BACTERIAL INFECTION AND BIOFILM FORMATION, EP 2 509 622
Antimicrobial-antibiofilm compositions and methods of use thereof, WO 2014134701
Hess et al., Antibacterial Synergy of Glycerol Monolaurate and Aminoglycosides in Staphylococcus aureus Biofilms, Antimicrob Agents Chemother. 2014 November; 58(11):6970-3.
Hess et al. The Natural Surfactant Glycerol Monolaurate Significantly Reduces Development of Staphylococcus aureus and Enterococcus faecalis Biofilm, Surgical Infections. October 2015, 16(5): 538-542. doi:10.1089/sur.2014.162.
Allen et al., The presence and impact of biofilm-producing staphylococci in atopic dermatitis, JAMA Dermatol. 2014 March; 150(3):260-5. doi: 10.1001/jamadermatol.2013.8627
Lieberman et al., A Review of Monolaurin and Lauric Acid: Natural Virucidal and Bactericidal Agents, Alternative and Complementary Therapies. December 2006, 12(6): 310-314. doi:10.1089/act.2006.12.310.
Stoodley, P., D. de Beer, and H. M. Lappin-Scott, Influence of Electric Fields and pH on Biofilm Structure as Related to the Bioelectric Effect, Antimicrob. Agents Chemother., 41(9):189 (1997).
69 Federal Register 34,937, C8, C10, and C12 Straight-Chain Fatty Acid Monoesters of Glycerol and Propylene Glycol; Exemption from the Requirement of a Tolerance (Jun. 23, 2004).

I claim:

1. A composition for cleaning, disinfecting or sterilizing a surface or object on which is disposed a microbial biofilm, the composition comprising:
(a) a cationic surfactant in an amount from about 1% w/v to about 5% w/v;
(b) one or more emulsifying agents in an amount from about 0.5% w/v to about 5% w/v;
(c) a biocide in an amount of at least about 0.1% w/v, wherein the biocide is a glycol monoester of the formula:
R₁OCH₂(OR₂)CH₂OR₃
wherein R₁, R₂ and R₃ are individually H or a C6 to C22 acyl group; and
(d) at least one weak acid in an amount from about 0.5% w/v to about 15% w/v, wherein:
(1) the at least one weak acid has a pH is less than about 3.5;
(2) the at least one weak acid comprises a first titration point pH; and
(3) the cationic surfactant has a pH of at least about 2 units greater than the first titration point pH of the at least one weak acid;
wherein a wetting layer is formed upon application of the composition on a surface or object comprising a microbial biofilm, wherein the wetting layer increases protonation of water to produce hydronium, and wherein the wetting layer increases delivery of the hydronium and the biocide to the microbial biofilm thereby disrupting the microbial biofilm.

2. The composition of claim 1, wherein the cationic surfactant is a fatty acid salt or a saponified organic acid, and wherein the at least one weak acid is selected from the group consisting of ascorbic acid, salicylic acid, citric acid, lactic acid, malic acid, tartaric acid and any combination thereof.

3. The composition of claim 2 wherein the cationic surfactant is potassium cocoate.

4. The composition of claim 1, wherein the one or more emulsifying agents are selected from the group consisting of sorbitan monolaurate, sodium stearoyl lactylate, polyoxyethylene (20) sorbitan monooleate and any combination thereof.

5. The composition of claim 1, wherein the glycol monoester is selected from the group consisting of glycerol monocaprylate, glycerol monocaprate, glycerol monolaurate, glycerol monomyristate and any combination thereof.

6. The composition of claim 1, wherein the composition is added to a cleaning formulation selected from the group consisting of toilet bowl cleaner, metal cleaner, metal brightener, rust stain remover, denture cleanser, metal descaler, general hard surface cleaner and disinfectant.

7. A method for cleaning, disinfecting or sterilizing a surface or object on which is disposed a microbial biofilm, the method comprising:
applying to the surface or object comprising the microbial biofilm a hyperprotonation composition, the hyperprotonation composition comprising:
(a) a cationic surfactant in an amount from about 1% w/v to about 5% w/v;
(b) one or more emulsifying agents in an amount from about 0.5% w/v to about 5% w/v;
(c) a biocide in an amount of at least about 0.1% w/v, wherein the biocide is a glycol monoester of the formula:
R₁OCH₂(OR₂)CH₂OR₃
wherein R₁, R₂ and R₃ are individually H or a C6 to C22 acyl group; and
(d) at least one weak acid in an amount from about 0.5% w/v to about 15% w/v, wherein:
(1) the at least one weak acid has a pH less than about 3.5;
(2) the at least one weak acid comprises a first titration point pH; and
(3) the cationic surfactant has a pH of at least about 2 units greater than the first titration point pH of the at least one weak acid;
wherein a wetting layer is formed upon application of the composition on the surface or object comprising the microbial biofilm, wherein the wetting layer increases protonation of water to produce hydronium, and wherein the wetting layer increases delivery of the hydronium and the biocide to the microbial biofilm thereby disrupting the microbial biofilm and cleaning, disinfecting or sterilizing the surface or object.

8. The method of claim 7, wherein:
(i) the cationic surfactant is a fatty acid salt or a saponified organic acid having a pH greater than about 8;
(ii) the at least one weak acid is selected from the group consisting of ascorbic acid, salicylic acid, citric acid, lactic acid, malic acid, tartaric acid and any combination thereof;
(iii) the one or more emulsifying agents are selected from the group consisting of sorbitan monolaurate, sodium stearoyl lactylate, polyoxyethylene (20) sorbitan monooleate and any combination thereof; and
(iv) the glycol monoester is selected from the group consisting of glycerol monocaprylate, glycerol monocaprate, glycerol monolaurate, glycerol monomyristate and any combination thereof.

9. The method of claim 7, wherein the surface or object is in a sports facility, fitness facility, stadium locker room, gymnasium, country club, restaurant, hospital, hotel or university.

10. The method of claim 7, wherein the surface or object is a fruit or vegetable.

11. The method of claim 7, wherein the surface or object is sprayed with the hyperprotonation composition or immersed in the hyperprotonation composition.

12. The method of claim 7, wherein the microbial biofilm comprises one or more microorganisms selected from the group consisting of gram positive bacterium, gram negative bacterium, virus, yeast, mold and any combination thereof.

13. The method of claim 7, wherein the applying comprises flood application, spray application, high pressure application, foam application or clean-in-place application.

14. The method of claim 7, wherein the applying is part of a sterilization sequence for medical devices.

15. The method of claim 7, wherein the hyperprotonation composition is contacted with the surface or object for a period of time of about 30 seconds to about 5 minutes, and wherein the method further comprises rinsing the hyperprotonation composition off of the surface or object after the period of time.

16. The method of claim 7, wherein the surface or object is selected from the group consisting of a piece of equipment, fabric, countertop, wall, door, toilet, shower stall, bathtub, sink, chair, food, locker, locker room, gymnasium floor and living tissue.

17. The method of claim 16, wherein the surface or object is a living tissue, and the hyperprotonation composition further comprises a pharmacologically acceptable carrier.

18. The method of claim 7, wherein the applying of the hyperprotonation composition to the surface or object produces a stable emulsified mixture in accordance with the hydrophilic-lipophilic balance system.

19. A composition for producing a hydronium engine on a microbial biofilm, the composition comprising:
(a) a cationic surfactant;
(b) one or more emulsifying agents;
(c) a biocide of the formula:
R₁OCH₂(OR₂)CH₂OR₃
wherein R₁, R₂ and R₃ are individually H or a C6 to C22 acyl group; and
(d) at least one weak acid having a pH less than or equal to 3.5 and having a first titration point that is at least about 2 units less than the pH of the cationic surfactant;
wherein application of the composition on the microbial biofilm produces an emulsion layer and a wetting layer, wherein the wetting layer increases protonation of water from the weak acid in the emulsion layer to produce hydronium, and wherein the wetting layer increases delivery of the hydronium and the biocide to the microbial biofilm thereby disrupting the microbial biofilm.

20. The composition of claim 19, wherein:
(a) the cationic surfactant comprises potassium cocoate in an amount from about 1% w/v to about 5% w/v;
(b) the one or more emulsifying agents is selected from the group consisting of glycerol monocaprylate, glycerol monocaprate, glycerol monolaurate, glycerol monomyristate, and any combination thereof, and wherein the one or more emulsifying agents are in an amount from about 0.5% w/v to about 5% w/v;
(c) the biocide is selected from the group consisting of glycerol monocaprylate, glycerol monocaprate, glycerol monolaurate, glycerol monomyristate, and any combination thereof, and wherein the biocide is in an amount of at least about 0.1% w/v; and
(d) the at least one weak acid is selected from the group consisting of ascorbic acid, salicylic acid, citric acid, lactic acid, malic acid, tartaric acid, and any combination thereof, and wherein the at least one weak acid is in an amount from about 0.5% w/v to about 15% w/v.
